(19) United States
(12) Patent Application Publication
(10) Pub. No.: US 2011/0262964 A1
(43) Pub. Date: Oct. 27, 2011

(54) Reagentless fluorescent biosensors comprising a designed ankyrin repeat protein module, rational design methods to create reagentless fluorescent biosensors and methods of their use

(76) Inventors: Hugues Bedouelle, Paris (FR); Elodie Brient-Litzler, Versailles (FR)

(21) Appl. No.: 12/933,363

(22) PCT Filed: Mar. 18, 2009
(86) PCT No.: PCT/IB2009/005308

§ 371 Date: Dec. 9, 2010

(30) Foreign Application Priority Data
Mar. 19, 2008 (EP) 08290262.8
Jul. 31, 2008 (EP) 08290742.9

(51) Int. Cl. C12P 21/00; C12P 21/02; C07K 14/00; C07K 17/00; G01N 33/566; C07K 2/00

(52) U.S. Cl. 435/69.1; 436/501; 530/300; 530/324; 530/350

(57) ABSTRACT

The present invention relates to reagentless fluorescent biosensors which comprise at least one ankyrin repeat and a fluorophore and are specific for at least one target; the method for preparing such reagentless fluorescent biosensors comprises the following steps: (a) identifying the residues (R₁) of the paratope of the biosensor by mutagenesis of all, or of a subset, of the residues of the biosensor, and determining variations in at least one measurable chemical or physical parameter of interaction with said at least one target; wherein said variations are due to each mutation or to groups of mutations; (b) selecting the cysteine residues, or the residues to be mutated to cysteine, from the residues (R₂) of the biosensor which are located adjacent to the residues of the paratope; (c) mutating by site-directed mutagenesis at least one of the residues (R₂) selected in (b) to a cysteine residue when said residue is not naturally a cysteine residue; and (d) coupling the Sγ atom of at least one cystein residue (R₂) obtained in (b) or in (c) to a fluorophore.

The present invention relates to reagentless fluorescent biosensors which comprise at least one designed ankyrin repeat protein (Darpins) and a fluorophore as well as to methods to generate reagentless fluorescent biosensors, in particular wherein no structural data exists of the biosensor in combination with its molecular target.

A molecular biosensor transforms a specific molecular binding event into a detectable signal and comprises several modules: a recognition module, which can also be called a receptor, can be of biological origin or biomimetic and which recognises at least one specific target such as an antigen, ligand or analyte during the binding event; a transduction module, which tranforms the recognition event into a measurable signal; and a means of evaluating the measurable signal data.

The recognition and transduction modules should be integrated into a compact device of molecular dimensions (Lowe 1984) and a molecular biosensor can function without additional reagents and provide quantitative analytical information and follow the concentration of its target, continuously. (Thevenot et al. 2001).

The physical nature of the measurable signal can be very diverse (Morgan et al. 1996). Fluorescence is an optical signal which allows one to detect molecular interactions with great sensitivity. The transduction is based on a variation of the fluorescence properties of the biosensor when it interacts with its analyte (Altschuh et al. 2006). The fluorescence of a protein biosensor can be intrinsic, e. g. provided by its component residues of tyrosine and tryptophan, or extrinsic, e. g. provided by the chemical coupling of fluorescent groups. The coupling of several fluorophores to a unique molecule of biosensor can be beneficial but is usually difficult to implement (Smith et al. 2005). Although intrinsic protein fluorescence can be used to study molecular interactions in purified experimental systems, extrinsic fluorescence is normally preferable to monitor specific interactions in complex media, without interference from other protein components (Foote and Winter 1992).

The changes of fluorescence that occur upon recognition between a reagentless fluorescent biosensor and its target, result from different interactions between the fluorescent group and its environment in the free and bound forms of the biosensor.

The binding of the target can occur in the neighborhood of the fluorescent group and directly modify its environment.

Alternatively, the binding of the target can induce a conformational change in the biosensor and thus cause an interaction between the fluorescent group and the receptor indirectly.

The inventors and others have used the first mechanism to create reagentless fluorescent biosensors from antibodies, first when the three-dimensional structure of the complex with their target is known and then in the absence of such a knowledge (Renard et al. 2002; Renard et al. 2003; Jespers et al. 2004; Renard and Bedouelle 2004). This work also formed the basis of WO2001/065258 which described such antibody based biosensor molecules.

Other groups have used the second mechanism to create biosensors from periplasmic binding proteins (de Lorimier et al. 2002).

In both cases, the receptor is modified such that it comprises a single cysteine residue which is normally introduced by site-directed mutagenesis in a pre-determined position of the receptor and a fluorophore is chemically coupled to this unique cysteine residue.

Antibodies are perfectly suited to provide the recognition module of biosensors since they can be directed against almost any target. The antibody is used in the form of a single-chain variable fragment or scFv. A residue of the single-chain variable fragment is identified which is in proximity to the target, when the single-chain variable fragment and target are in a complex. The selected residue is changed into a cysteine by site-directed mutagenesis. A fluorophore is chemically coupled to the mutant cysteine. The binding of the target shields the fluorophore from the solvent and can therefore be detected by a change of fluorescence.

Antibodies however have several intrinsic limitations. The single-chain variable fragments, which serve as the starting molecules for the construction of biosensors, often have insufficient conformational stability and limited half-lives to be suitable for prolonged use or use in harsh conditions. They contain two disulfide bonds, one in each variable domain. Therefore, when produced in a prokaryote they must be exported into the oxidizing medium of the bacterial periplasm to allow permissive conditions for the formation of their disulfide bonds and their folding in a functional form. The necessity of periplasmic expression limits the yield of total peptide production in prokaryotes significantly. In addition the mutant cysteine in the single-chain variable fragment to which the fluorophore is chemically coupled, often needs to be reactivated by a mild reduction before coupling. This reduction partially attacks the disulfide bonds of the fragment and further decreases the production yield of fluorescent single-chain variable fragment conjugates.

The general problems of expression and stability that are encountered with most recombinant antibodies have slowed down their exploitation and these problems have led several groups to develop alternative families of target binding proteins, by engineering proteins that have a stable polypeptide scaffold, are devoid of cysteine residues and disulfide bonds and consequently are well expressed in Escherichia coli. One new family of target binding proteins, Darpins, have been shown able to replace antibodies in many of their applications (Mathonet and Fastrez 2004; Binz et al. 2005).

The family of the Designed Ankyrin Repeat Proteins (Darpins) is a well characterized artificial family of target binding proteins. The ankyrin repeats are present in thousands of proteins from all phyla and involved in recognitions between proteins (Mosavi et al. 2004; Li et al. 2006). Consensus sequences of these modules have been established and the corresponding consensus proteins have been shown to possess remarkable biophysical properties (Mosavi et al. 2002; Binz et al. 2003; Kohl et al. 2003).

Combinatorial libraries of Darpins have been generated by randomization of residues that potentially belong to the paratope (target binding site) and the assemblage of a random number of ankyrin modules between defined N- and C-terminal modules (Binz et al. 2003). These libraries were used to select Darpins that bound specific protein targets, using ribosome display (Zahnd et al. 2007).

In particular the Inventors have now developed a new type of reagentless biosensors which incorporate the advantages of Darpins; the inventors have also developed methods to design and produce such new reagentless biosensors.

This new class of Darpin based reagentless biosensors overcome the problems associated with antibody based reagentless biosensors, such as poor physicochemical properties and complex production regimes. In addition the inventors have unexpectedly found that the rate of successful creation of Darpin biosensors is greater than with their previous work using antibodies. Also the inventors have found that Darpin based RF (Reagentless fluorescent) biosensors according to the current invention have higher sensitivity in comparison to the RF biosensors based upon antibodies and antibody fragments that they previously developed.

The inventors have therefore developed a novel method to generate RF biosensors and describe herein such novel RF biosensors and rules for the design of RF biosensors from Darpins when the three-dimensional structure of the complex with the ligand is known or unknown.

Therefore the present invention relates to a reagentless peptide biosensor for at least one ligand, comprising:

at least one ankyrin repeat module;

at least one cysteine residue coupled to a fluorophore.

In the current Application an ankyrin repeat module is one which consists of one or more ankyrin repeat.

The ankyrin repeat, a 33-residue sequence motif, was first identified in the yeast cell cycle regulator Swi6/Cdc10 and the Drosophila signalling protein Notch (Breeden and Nasmyth 1987), and was eventually named after the cyto-skeletal protein ankyrin, which contains 24 copies of this repeat (Lux et al. 1990). Subsequently, ankyrin repeats have been found in many proteins spanning a wide range of functions.

The individual ankyrin repeats in the ankyrin repeat module can be identical or different. Each of these ankyrin repeats may each comprise a fluorophore or not and in each ankyrin repeat the flurophore may be attached to the same or a different residue within each of the ankyrin repeats.

In particular the cysteine residue is present at a position of the biosensor whose solvent accessible surface area is altered when said biosensor binds to said at least one ligand but which does not directly interact therewith.

The finding by the inventors that Darpins can be used to generate reagentless fluorescent biosensors and in particular that these Darpin based biosensors can compare and in some cases out perform, in terms of sensitivity and other characterisitics, the previous antibody based biosensors they generated was unexpected. Darpins and antibodies are not structurally similar molecules, antibodies being the main mediator of acquired immunity in higher animals whereas Darpins are an artificial class of protein based upon the ubiquitous (in nature) ankyrin domain. With reference to Binz et al., 2004 a number of specific features of Darpins are listed, these include a rigid body structure (p. 580, middle of 2^{nd} paragraph), a high stability under denaturing conditions such as heat or chemical reactants (D2, p. 576, 1^{st} column end) and a buried surface area (that is the surface buried away from solvent when two proteins or subunits associate to form a complex) in combination with their target which is lower than usual for antibody-target interactions (p. 579, 1^{st} column middle of last paragraph).

Therefore several technical differences exist between antibodies and Darpins. Importantly some of the technical differences between Darpins and antibodies pointed out in Binz et al., 2004 would probably have led the man skilled in the art to consider Darpins inferior to antibodies for the preparation of peptide biosensors according to the present invention.

Surprisingly the inventors have now proven that these inherent characteristics of Darpins do not affect the performance of Darpin based reagentless biosensors and also that such biosensors share the attractive properties of Darpins, namely their ease of expression, purification and handling.

The inventors therefore provide a new class of reagentless biosensor which has the advantages of a reagentless biosensor, namely a biosensor which can function without additional reagent and can provide quantitative analytical information and follow the concentration of its analyte continuously together with the more robust bio/physico-chemical properties of Darpins and without any apparent loss of sensitivity or binding affinity. They have validated this new class of RF biosensors with the known Darpin DarpOff7, a Darpin which is directed against the MalE protein (Binz et al. 2004).

The inventors have shown that several variants of such Darpin based biosensors work using the MalE protein from Escherichia coli as a model target.

Such reagentless fluorescent biosensors can be used in different formats: in solution, in the form of protein chips, or at the tip of optical micro- or nano-fibers. They could be used for the continuous quantification of antigens in complex mixtures, without any prior labelling of the proteins under analysis.

In healthcare, they could be used for the bed side monitoring of patients, the controlled continuous delivery of drugs, the control of artificial organs, some diagnostics, in situ measurements during surgical operations and the detection of doping drugs.

In industry, they could be used for the monitoring of reactions and processes, food control and pharmacokinetic studies.

In environmental protection/monitoring and civil or military defence, they could be used for the monitoring of pathogenic, toxic or polluting agents.

In fundamental research, they could be used in proteomics, for the profiling of cells, tissues or body fluids; in the biology of single cells, to continuously measure the concentration of an antigen within a single living cell; in neuro-chemistry and neuro-sciences, to measure the intra-cerebral concentration of neuro-peptides in response to external stimuli.

In particular the reagentless biosensor may be derived from a parental binding protein for said ligand.

In the current Application a parental binding protein refers to any protein known or suspected to have binding affinity for a given ligand and from which the binding portion of this protein can be isolated and used in the construction of a reagentless biosensor according to the current invention.

In particular each ankyrin repeat is a 30 to 35 residue polypeptide comprising a canonical helix-loop-helix-beta hairpin/loop fold structure.

In particular this biosensor comprises at least one ankyrin repeat which consists of SEQ ID NO: 30 or SEQ ID NO: 7 or a sequence of at least 60% similarity therewith.

These percentages of sequence similarity defined herein were obtained using the BLAST program (blast2seq, default parameters) (Tatutsova and Madden, FEMS Microbiol Lett., 1999, 174, 247-250).

SEQ ID NO: 30 and SEQ ID NO: 7 represent consensus sequences of the ankyrin repeat.

Such percentage sequence similarity is derived from a full length comparison with SEQ ID NO:30 or SEQ ID NO:7, as detailed herein; preferably these percentages are derived by calculating them on an overlap representing a percentage of length of SEQ ID NO: 30 or SEQ ID NO: 7.

In particular the biosensor comprises at least one ankyrin repeat which has at least 80% similarity with SEQ ID NO: 30 or SEQ ID NO: 7.

In particular the biosensor comprises at least one ankyrin repeat which has at least 95% similarity with SEQ ID NO: 30 or SEQ ID NO: 7.

In particular the biosensor according to the current invention has a fluorophore coupled to an ankyrin repeat of the ankyrin repeat module at a position selected from:
(i) residues 2, 3, 5, 13, 14, 26 and 33; or
(ii) residues 1, 4, 6, 12, 15, 25, 27, 32,

the residues being changed to cysteine residues if they are not already cysteine residues.

In particular the biosensor according to the present invention has a fluorophore coupled to one residue of SEQ ID NO: 30 or SEQ ID NO: 7, selected from the sets (i) and (ii) of the residues above.

In particular the biosensor or its parental binding protein may comprise at least an N-terminal capping ankyrin repeat and/or a C-terminal capping ankyrin repeat.

In particular the N-terminal capping ankyrin repeat consists of SEQ ID NO: 8 or SEQ ID NO: 23 and the C-terminal capping ankyrin repeat consists of SEQ ID NO: 10 or SEQ ID NO: 24.

In particular in the biosensor according to the present invention the at least one cysteine residue is either present in said biosensor or is substituted with another suitable residue. Wherein the at least one cysteine residue or the substituted residue has a solvent accessible surface area which is altered when the biosensor binds to the ligand, but which does not directly interact directly therewith.

In particular the residue forms an indirect contact with the ligand via at least one water molecule.

Alternatively the residue does not contact the ligand, neither directly nor indirectly.

In particular the fluorophore is selected from the group consisting of: 6-acryloyl-2-dimethylaminophtalene(acrylodan), 4-chloro-7-nitrobenz-2-oxa-1,3-diazole (CNBD), 5-iodoacetamidoflurescein (5-IAF), (N-((2-(iodoacetoxy)ethyl)-N-methyl)amino-7-nitrobenz-2-oxa-1,3-diazole (IANBD ester), Cy3, Cy5 or a fluorophore having an aliphatic chain of 1 to 6 carbon atoms.

A fluorophore, is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore.

In the present Patent Application when one of the variously described biosensors is in combination with a flurophore these will be described using the following non-clementure X(YZ), wherein X is the name of the Darpin from which the biosensors has been generated for instance DarpOff7, Y is the name of the residue in the biosensor which has been changed to cysteine and to which the fluorophore is attached for instance (N45 . . . and Z is the name of the fluorophore attached to the biosensor for instance . . . ANBD), ANBD being the derivative of IANBD which attaches to the cysteine residue. The full name of this biosensor being DarpOff7(N45ANBD).

In particular the biosensor is in soluble form.

In particular the biosensor is immobilized on a suitable solid support.

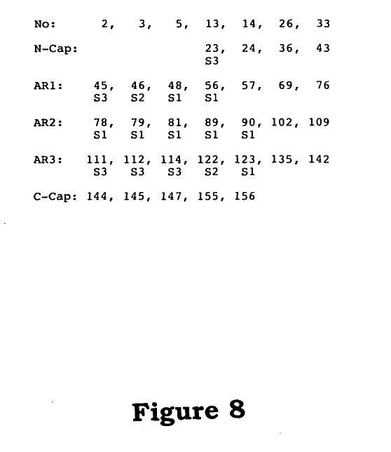

The present invention also relates to a biosensor which consists of SEQ ID NO: 28 in which one of residues 23, 45, 46, 53, 111, 112, 114, 122, 123 and 125 has been substituted with a cysteine residue and coupled to a fluorophore. The relationship of these residues to the ankyrin repeat consensus sequence is shown in FIG. 8.

The present invention also relates to a protein-based chip, characterized in that it consists of a solid support on which at least one biosensor as described in the current Patent Application is immobilized.

The present invention also relates to a solution comprising at least one biosensor as described in the current Patent Application.

The present invention also relates to an optical fibre comprising at a first end thereof at least one biosensor as described in the current Patent Application and comprising at a second end thereof means to attach the optical fibre to a device configured to receive and interpret the output of the at least one biosensor.

The present invention also relates to a method for producing biosensors as described in the current Patent Application, characterized in that it comprises the following steps:

(a) selecting at least one residue of the biosensor by searching for the residues which have a solvent accessible surface area (ASA) which is modified by the binding of said at least one ligand, when use is made of spheres of increasing radius of 1.4 to 30 Å, for the molecule of said solvent; and which (i) are in contact with said ligand via a water molecule, or (ii) do not contact said ligand;

(b) mutating by site-directed mutagenesis at least one of the residues selected in (a) to a Cys residue when said residue is not naturally a Cys residue, and

(c) coupling the Sγ atom of at least one Cys residue obtained in (a) or in (b) to a fluorophore.

In particular the preparation method is characterised in that the biosensor comprises at least a portion of a parental protein known to bind the ligand.

In particular the preparation method is characterized in that, prior to step (a), it comprises a step of modelling the biosensor or its parental protein and/or the ligand and/or the biosensor/parental protein-ligand complexes.

In particular this modelling may be either by means of ab initio protein structure modelling programmes such as MODELLER or swissmodeller; or comparative protein modelling using previously solved structures as starting points. Alternatively using 3D models derived from protein crystallography, NMR or other means.

These above methods are limited by the need for structural data of the Darpin in complex with its target, from which the necessary calculations can be made as to which residues are suitable targets for mutation to a cysteine residue and coupling with a fluorophore.

Therefore for this class of reagentless fluorescent biosensor which comprises at least one designed ankyrin repeat and a fluorophore to be generally useful, it is necessary for a more generalized/rational design methodology to be developed for use with Darpins of unknown structures because structural data are rarely available and in the case of a newly generated Darpin with a selected specificity such structural data will not be available.

Seeing these problems with the prior art the inventors have according to a further aspect of the present invention developed a rational approach to the choosing of sites for the coupling of a fluorophore with any Darpin and thus creating reagentless fluorescent biosensors from this parental Darpin even when the structure of the complex of the Darpin with its target is unknown.

Therefore according to this aspect of the present invention there is provided a method for preparing reagentless fluorescent biosensors which comprise at least one ankyrin repeat and are specific for at least one target, characterized in that it comprises the following steps:

(a) identifying the residues (R₁) of the paratope of the biosensor by mutagenesis of all, or of a subset, of the residues of the biosensor, and determining variations in at least one measurable chemical or physical parameter of interaction with said at least one target,

wherein said variations are due to each mutation or to groups of mutations;

(b) selecting the cysteine residues, or the residues to be mutated to cysteine, from the residues (R₂) of the biosensor which are located adjacent to at least one residue of the paratope; and/or selecting the cysteine residues, or the residues to be mutated to cysteine, from the residues (R₃) which do not form part of the paratope and which were mutated in step (a);

(c) mutating by site-directed mutagenesis at least one of the residues (R₂) and/or (R₃) selected in (b) to a cysteine residue when said residue is not naturally a cysteine residue; and

(d) coupling the Sγ atom of at least one cysteine residue (R₂) and/or (R₃) obtained in (b) or in (c) to a fluorophore.

The inventors have therefore provided a new rational design method which can be used to adapt any existing or newly generated target specific molecule which comprises at least one ankyrin domain in the complete absence of any structural data concerning the biosensor and its target.

In this Patent Application the target can be any naturally occurring or synthetic substance or component thereof against which the biosensor has specific binding affinity.

In this Patent Application the Paratope is defined as one or more residues the positioning and biochemical properties of which in the biosensor make a significant contribution to target recognition and binding and the alteration of which either by their removal or due to a change in their biochemical properties decreases biosensor-target interactions.

The method essentially comprises two stages, firstly the identification of one or more of a first set of residues (R₁) of the biosensor which are involved in target recognition and binding, called the paratope herein. Secondly this rational design method involves the modification to cysteine of at least one of a second set of residues (R₂) which are adjacent to one or more of the first set (R₁) and the coupling of the modified biosensor to a fluorophore at this cysteine. Also in this second step residues identified as not being involved in the paratope (R₃) in step (a), can also be selected for alteration to cysteine and coupled with a flurophore at this cysteine.

The inventors have shown that it is not necessary to couple the fluorophore to a residue which is important for target interaction, because the fluorophore group will hinder said interaction Darpin-target.

Nevertheless it is best to target the coupling of the fluorophore to a residue neighbouring an antigen (or target) binding residue as it is then likely that the recognition and binding of the target and biosensor will modify the environment of the fluorophore and induce a detectable variation in fluorescence.

Based on this principle the method seeks to identify at least one residue which is functionally important for interaction with the target and from this to go on to identify a residue which is adjacent to this functionally important residue, for example by reference to its sequence or to a canonical structure.

The inventors have also shown however that other residues (R₃) identified as not being important to antigen binding in the first stage of the rational design method can also potentially be used to couple a fluorophore to the biosensor and so generate a reagentless fluorescent biosensor.

In particular wherein said at least one measurable chemical or physical parameter is selected from the group: the equilibrium constant (K_{D}) between said biosensor and said at least one target; the dissociation (K_{off}) and/or association (kₒₙ) rate constants for said biosensor and said at least one target; variation of free energy of interaction (ΔΔG) between said biosensor and said at least one target; variation of resonance signal at equilibrium (R_{eq}) between said biosensor and said at least one target or any other means of measuring the biosensor/target interaction.

To determine which of the residues of the biosensor constitute the first set (R₁) and form the paratope, a number of specific measurements can be made to characterize biosensor-target interactions. These measurements include determining the equilibrium constant (K_{D}) between said biosensor and said at least one target; the dissociation (K_{off}) and/or association (kₒₙ) rate constants for said biosensor and said at least one target; variation of free energy of interaction (ΔΔG) between said biosensor and said at least one target; variation of resonance signal at equilibrium (R_{eq}) between said biosensor and said at least one target. In the present Patent Application the inventors provide several examples of how these measurements can be determined by various experimental means.

As stated above designed ankyrin repeat proteins (Darpins) can be directed against any target and have favourable properties of recombinant expression, solubility and stability. They are isolated from combinatorial libraries that are generated by randomization of the residues that potentially belong to the target binding site in a consensus ankyrin module, and assemblage of a random number of repeats.

Therefore the possibility of obtaining from any Darpin, a fluorescent conjugate which responds to the binding of the target by a variation of fluorescence, which would have numerous applications in micro- and nano-analytical sciences is now provided by the rational design methodology of the present invention.

The ankyrin repeat, a 33-residue sequence motif, was first identified in the yeast cell cycle regulator Swi6/Cdc10 and the Drosophila signalling protein Notch (Breeden and Nasmyth 1987), and was eventually named after the cyto-skeletal protein ankyrin, which contains 24 copies of this repeat (Lux et al. 1990). Subsequently, ankyrin repeats have been found in Many proteins spanning a wide range of functions. If the biosensor comprises more than one ankyrin repeat the individual ankyrin repeats in the ankyrin repeat module can be identical or different. Each of these ankyrin repeats may each comprise a fluorophore or not and in each ankyrin repeat the flurophore may be attached to the same or a different residue.

The inventors have tested and validated this approach with DarpMbp3_{—}16, a Darpin which comprises two ankyrin repeats and is directed against the same target as DarpOff7, i.e. the MalE protein of E. coli (Binz et al. 2004).

In particular the reagentless biosensor may be derived from a parental binding protein for said target.

This parental binding protein can be a Darpin generated according to the methodologies described for instance in Binz et al. 2004 or a native protein with a specific affinity for a particular target or one or more isolated ankyrin repeats from such a native protein.

In the current Application a parental binding protein refers to any protein known or suspected to have binding affinity for a given ligand and from which the binding portion of this protein can be isolated and used in the construction of a reagentless biosensor according to the current invention.

In particular each ankyrin repeat is a 30 to 35 residue polypeptide comprising a canonical helix-loop-helix-beta hairpin/loop fold structure.

In particular in step (b) the selected adjacent residues (R₂) are residues −1 and +1 along the peptide backbone relative to at least one residue of the paratope.

Alternatively in step (b) the selected adjacent residues (R₂) are in Van-Der-Waals contact with at least one residue of the paratope.

In particular prior to step (a) the nonessential Cys residues of the biosensor are substituted with Ser or Ala residues by site-directed mutagenesis.

In particular, in step (d), said fluorophore is selected from the group consisting of: IANBD, CNBD, acrylodan, 5-iodoacetamidofluorescein or a fluorophore having an aliphatic chain of 1 to 6 carbon atoms.

A fluorophore is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore.

In particular the at least one ankyrin repeat comprises a number of framework residues and a number of variable residues, and said subset of residues of step (a) which are mutated, comprise at least one of said variable residues.

The designed ankyrin repeats which are used to generate new target specific Darpins are normally based upon a consensus sequence in which some of the residues are fixed, known as framework residues, so as to provide the characteristic helix-loop-helix-beta hairpin/loop fold structure and some of the residues are varied, known as variable residues, in a random or semi random fashion so as to alter the binding properties of the Darpin. The inventors have found that by focussing efforts upon these variable residues the method works more efficiently.

For instance in the experiments described in the current Patent Application in the two designed ankyrin repeats which DarpMbp3_{—}16 comprises, there are twelve residues that correspond to fully randomized positions in the parental library, were individually changed into cysteine by mutagenesis, and then chemically coupled with an environment sensitive fluorophore.

In particular the at least one ankyrin repeat consists of SEQ ID NO: 7.

Most particularly said subset of residues of step (a) which are mutated are selected from residues 2, 3, 5, 13, 14, 26 and 33 of SEQ ID NO: 7.

In particular the biosensor comprises at least an N-terminal capping ankyrin repeat and/or a C-terminal capping ankyrin repeat.

In particular the N-terminal capping ankyrin repeat consists of SEQ ID NO: 8 or SEQ ID NO: 23, and the C-terminal capping ankyrin repeat consists of SEQ ID NO: 10 or SEQ ID NO: 24.

In particular said subset of residues (R₁) of step (a) also comprises residue 43 of SEQ ID NO: 8 or SEQ ID NO: 23.

In particular prior to step (d) the mutated biosensor obtained in step (c) is subjected to a controlled chemical reduction.

In particular after step (d), the method comprises an additional step (e) of:

(e) purifying the biosensor of step (d).

In particular after step (e), the method comprises an additional step (f) of:

(f) (i) measuring the equilibrium constant (K_{D}) between said purified biosensor and said at least one target, or the dissociation (K_{off}) and association (kₒₙ) rate constants for said biosensor and said at least one target; and

(ii) measuring the fluorescence variation of said biosensor between a free and target bound state; and

(g) determining the sensitivity (s) and/or relative sensitivity (sᵣ) of said biosensor from the measurements of step (f) (i) and (ii).

Based upon experimental data concerning the interaction of the purified fluorescent biosensor with its target and the fluorescence characteristics of this interaction, it is possible to determine the sensitivity of the biosensor which is an important feature of such biosensors and determines what types of role the biosensor can be used in.

In particular the biosensor may be purified in a soluble form.

In particular after step (d) or step (e) or step (f), the method comprises an additional step of immobilizing said biosensor on a solid support.

According to a further aspect of the present invention there is provided a method, wherein said biosensors comprise at least two ankyrin repeats and is characterized in that it comprises the following replacement steps:

(a1) identifying the paratope of a first ankyrin repeat by scanning mutagenesis of the set or of a subset of the residues of said first ankyrin repeat, and determining the variations in the parameters of interaction with the ligand (K_{D}, kₒₙ, k_{off}, ΔΔG, R_{eq}) which are due to each mutation or to limited groups of mutations;

(b1) selecting the Cys residues, or the residues to be mutated into cysteine, from the residues of a second ankyrin repeat which are (i) equivalent to the residues of the paratope, (ii) are located in proximity of the residues of the paratope of said first ankyrin repeat or (iii) are in spatial proximity with the paratope of said first ankyrin repeat;

(c1) mutating by site-directed mutagenesis at least one of the residues selected in (b1) to a Cys residue when said residue is not naturally a Cys residue; and

(d1) coupling the Sγ atom of at least one Cys residue obtained in (b1) or in (c1) to a fluorophore.

The present invention provides also a method to create bivalent or bifunctional Darpins dimers comprising two or more ankyrin repeats linked by disulfide bonds. Such bifunctional Darpins enlarge the potential functionalities of Darpins. In particular, two or more homologous ankyrin repeats, linked by a disulfide bond, can generate an avidity effect for a multivalent target; or two or more heterologous ankyrin repeats can allow one hetero-dimeric or -multimeric molecule to bind two or more targets simultaneously. Preferably, the cysteine residue, involved in forming the disulfide bond between two or more ankyrin repeats should be outside of the paratopes as to not interfere with the Darpins/targets interactions.

In particular the at least two ankyrin repeats are homologous.

Alternatively the at least two ankyrin repeats are heterologous.

According to another aspect of the present invention there is provided a biosensor produced according to a method of the first or second aspect of the present invention.

In particular the biosensor, comprises a peptide sequence selected from the group: SEQ ID NO: 11, SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39.

According to a further aspect of the present invention there is provided a protein-based chip, characterized in that it consists of a solid support on which at least one biosensor of the present invention or produced according to a first or second aspect of the present invention.

According to another aspect of the present invention there is provided a solution comprising at least one biosensor as per the third aspect of the present invention or produced according to a first or second aspect of the present invention.

According to another aspect of the present invention there is provided an optical fibre comprising at a first end thereof at least one biosensor as per the third aspect of the present invention or produced according to a first or second aspect of the present invention and comprising at a second end thereof means to attach said optical fibre to a device configured to receive an interpret the output of said at least one biosensor.

For a better understanding of the invention and to show how the same may be carried into effect, there will now be shown by way of example only, specific embodiments, methods and processes according to the present invention with reference to the accompanying drawings in which:

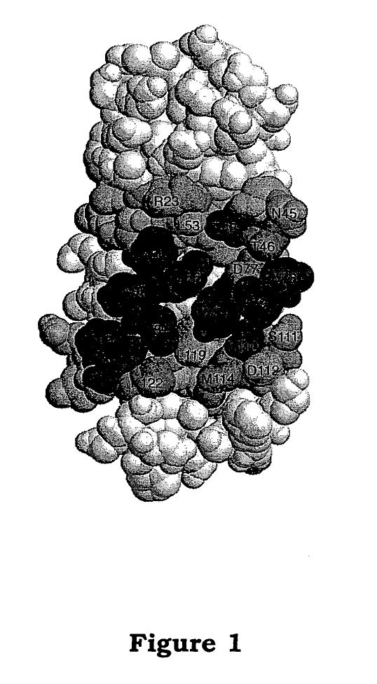

FIG. 1. Shows the positions of the coupling sites in the structure of DapOff7.

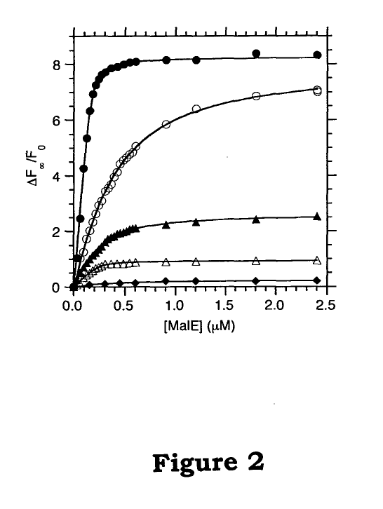

FIG. 2. Shows the titration of DarpOff7 conjugates by MalE, monitored by fluorescence.

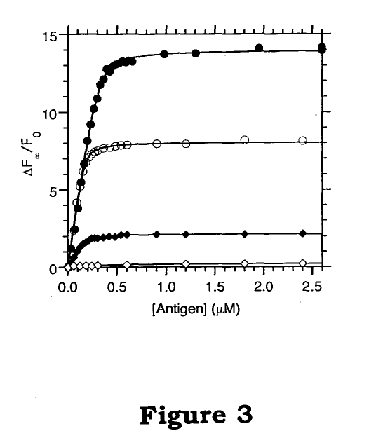

FIG. 3. Shows the selectivity and specificity of the fluorescence signal for the DarpOff7(N45ANBD) conjugate.

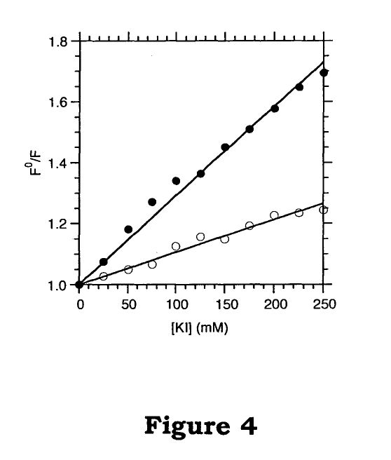

FIG. 4. Shows the quenching of the DarpOff7(N45ANBD) fluorescence by KI.

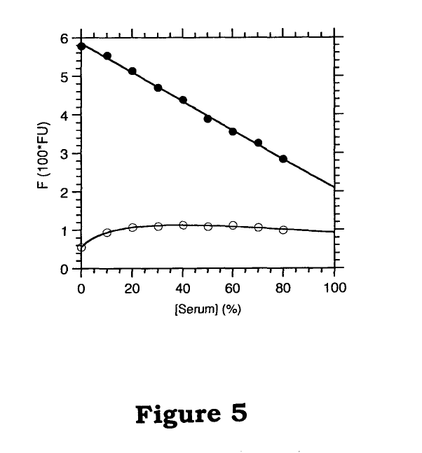

FIG. 5. Shows the effects of the concentration in serum on the fluorescence signals for the DarpOff7(N45ANBD) conjugate.

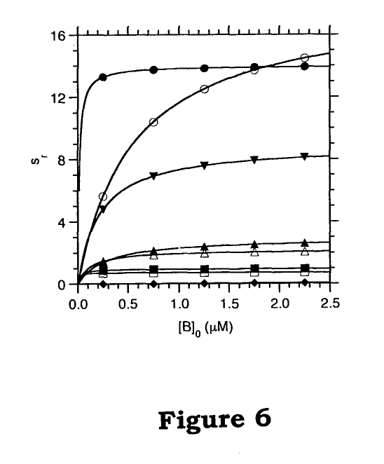

FIG. 6. Shows the ranking of the DarpOff7 conjugates according to their relative sensitivities sᵣ at 25° C. in buffer L1.

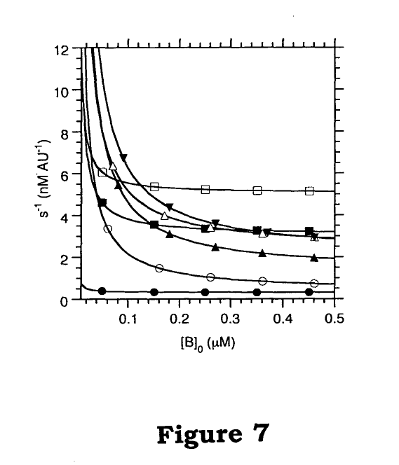

FIG. 7. Shows the ranking of the DarpOff7 conjugates according to their lower limit of detection at 25° C. in buffer L1.

FIG. 8. Shows the relative positions of the coupling sites in the ankyrin repeats.

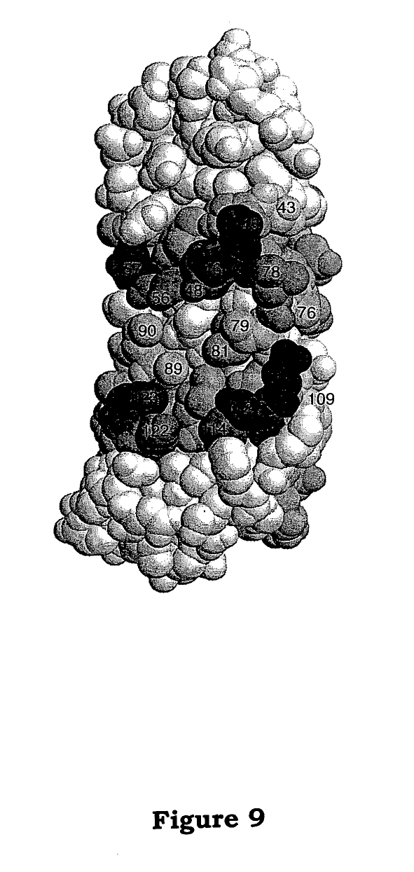

FIG. 9. shows the randomized positions in the crystal structure of the consensus DarpE3_{—}5. The ankyrin repeats are represented in alternating light grey and dark grey, with the N-cap on top.

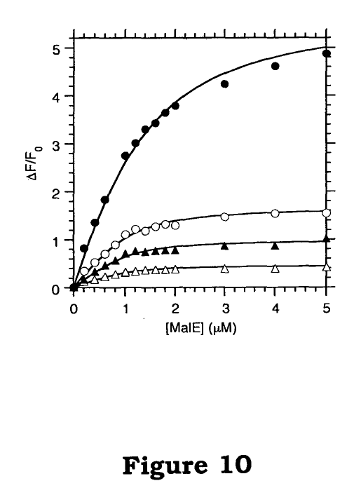

FIG. 10. shows the titration of DarpMbp3_{—}16 conjugates by MalE, monitored by fluorescence.

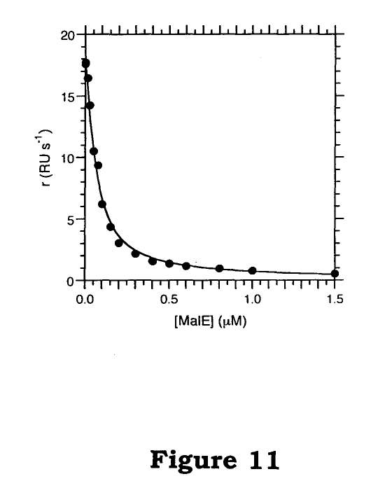

FIG. 11. shows the determination of the dissociation constant between DarpMbp3_{—}16(wt) and MalE by competition Biacore in solution.

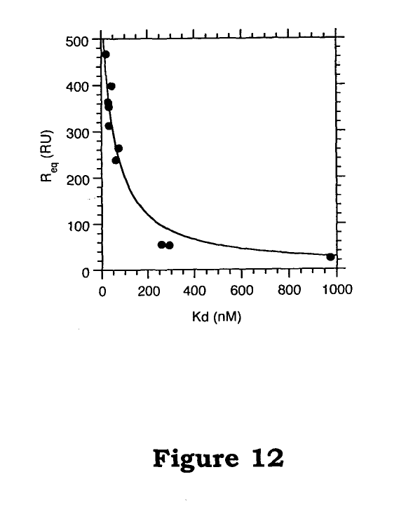

FIG. 12. shows the relation between R_{eq} and K_{d} for the interaction between the mutant DarpMbp3_{—}16 and MalE.

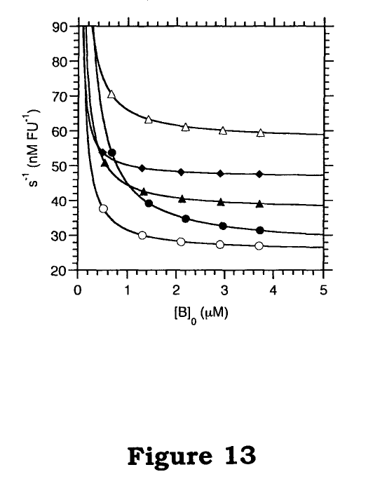

FIG. 13. shows the ranking of DarpMbp3_{—}16 conjugates according to their lower limit of detection at 25° C. in buffer M1.

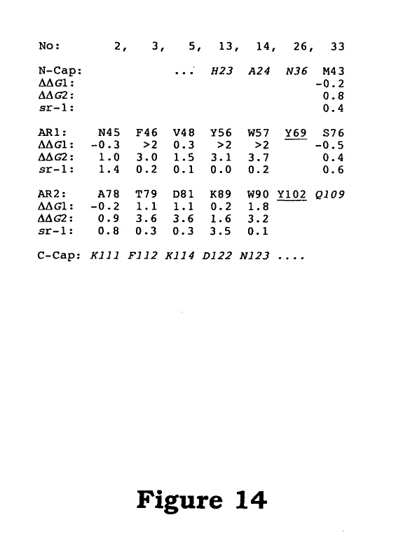

FIG. 14. shows the relative positions of the coupling sites in the ankyrin repeats. AR1 and AR2, ankyrin repeats 1 and 2 respectively.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Materials and Methods

### 1.1 Analysis of the Structural Data

The crystal structure of the complex between DarpOff7 and MalE (PDB 1SVX) was analyzed with the What If program (Vriend 1990).

The solvent accessible surface area (ASA) were calculated with the ACCESS routine and a radius of the solvent sphere equal to 1.4 Å (Ångström). The contact residues between DarpOff7 and MalE were identified with the ANACON routine, using extended Van der Waals radii as described (Rondard and Bedouelle 1998). Water molecules bridging DarpOff7 and MalE were identified with the subroutine NALWAT. The three-dimensional structures of the cysteine mutants of DarpOff7 were modeled with the mutation prediction program of the What If web interface (http://swift.cmbi.kun.nllwhatif/).

### 1.2 Materials

LB medium, the Escherichia coli strains XL1-Blue (Bullock et al. 1987) and AVB99 (Smith et al. 1998); the plasmid vector pQE30 (Qiagen) and the recombinant plasmids pQEMBP (SEQ ID NO: 3), pAT224 (SEQ ID NO: 4) pQEOFF7 (SEQ ID NO: 26) and the DarpOff7 (SEQ ID NO: 27) and pQEmbp3_{—}16 (SEQ ID NO: 1) (Binz et al. 2004) are as described in the references cited. pQEMBP (SEQ ID NO: 6) codes for the maltose binding protein MalE from E. coli. pAT224 (SEQ ID NO: 7) codes for a hybrid bt-MalE between a peptide that can be biotinylated in vivo by E. coli, and MalE. DarpOff7 (SEQ ID NO: 26) codes for a Darpin, DarpOff7 (SEQ ID NO: 28), directed against MalE.

pQEMBP codes for the maltose binding protein MalE from E. coli.

pAT224 codes for a hybrid bt-MalE which comprises a peptide that can be biotinylated in vivo by E. coli and MalE.

PQEMbp3_{—}16 encodes the nucleotide sequence of DarpMbp3_{—}16 (SEQ ID NO: 1) which in turn encodes the peptide DarpMbp3_{—}16 (SEQ ID NO: 2), a Darpin which is directed against MalE. All the recombinant proteins carry a hexahistidine tag (H6).

The Darpin, DarpMbp3_{—}16 consists of four ankyrin repeats, a N-terminal capping ankyrin repeat (SEQ ID NO: 8), two designed ankyrin repeats (SEQ ID NO: 9) and a C-terminal capping ankyrin repeat (SEQ ID NO: 10). These N- and C-terminal capping terminal ankyrin repeats are based upon consensus N- and C-terminal capping terminal ankyrin repeats of SEQ ID NO: 23 and SEQ ID NO: 24 respectively. The function of these terminal repeats is to shield the hydrophobic core of the final protein.

The residues which are varied in the designed ankyrin repeat domains of DarpMbp3_{—}16 are:

### Asp Xaa Xaa Gly Xaa Thr Pro Leu His Leu Ala Ala Xaa Xaa Gly His Leu Glu Ile Val Glu Val Leu Leu Lys Zaa Gly Ala Asp Val Asn Ala Xaa(SEQ ID NO: 7)

Wherein when generating the initial Darpin library, Xaa can represent any natural amino acid except for glycine, cysteine or proline; and Zaa can be any one of the amino acids asparagine, histidine or tyrosine.

The inventors targeted all the fully randomized positions of DarpMbp3_{—}16 and neglected residues 69 and 102, which are only partially randomized and are located on a different side of the molecule as predicted from the structure of the canonical Darp3_{—}5 (FIG. 9).

DarpMbp3_{—}16 was generated using a library comprising a random number of a consensus ankyrin repeat sequence (SEQ ID NO: 7) which is variable at positions 2, 3, 5, 13, 14 and 33.

Hence DarpMbp3_{—}16 comprises two copies of this consensus ankyrin repeat sequence between a C- and N-terminal capping ankyrin repeat. The final residue of the N-terminal capping ankyrin repeat (SEQ ID NO: 8) is also variable at its final residue this being the equivalent of residue 33 of the designed ankyrin repeat (SEQ ID NO: 7) in the N-terminal capping ankyrin repeat (SEQ ID NO: 8).

Buffer H was 500 mM NaCl, 50 mM Tris-HCl (pH 7.5); buffer M1, 150 mM NaCl, 50 mM Tris-HCl (pH 7.5); buffer L1, 50 mM NaCl, 20 mM Tris-HCl (pH 7.5); buffer M2, 0.005% (v/v) P20 surfactant (Biacore) in buffer M1; buffer L2, 0.005% (v/v) P20 surfactant in buffer L1; buffer M3, 5 mM dithiothreitol (DTT) in buffer M2. Ampicillin was used at a concentration of 100 μg/mL and chloramphenicol at 10 μg/mL. Phosphate buffer saline (PBS), calf serum and DTT were purchased from Sigma, N-((2-(iodoacetoxy)ethyl)-N-methyl)amino-7-nitrobenz-2-oxa-1,3-diazole (IANBD ester) from Invitrogen. A stock solution of the IANBD ester was made at a concentration of 10 mg/mL in dimethylformamide. Ampicillin was used at a concentration of 100 μg/mL and chloramphenicol at 10 μg/mL.

### 1.3 Mutagenesis and Protein Production, Purification and Characterization

Changes of residues were constructed in the DarpOff7 protein (SEQ ID NO: 28) at the genetic level, by mutagenesis of pQEOff7 open reading frame (SEQ ID NO: 26) with the Quickchange II site directed mutagenesis kit (Stratagene). As Darpins consist of repeated modules of the ankyrin repeat polypeptide and encoded by similarly repeated segments of DNA, the mutagenic primers were designed so that their 3′-nucleotide or the preceding nucleotide was specific of the targeted segment and their elongation by DNA-polymerase could not occur on another repeated segment. Mutation K122C could not be obtained in this way. To obtain this residue change the inventors therefore used the degeneracy of the genetic code to design a mutant allele of the off7 gene that was devoid of extensive repetitions. The mutant allele, off71 (SEQ ID NO: 29), was synthesized by Genecust (Evry, France) and used to construct mutations K68C and K122C.

Darpins are formed of repeated polypeptidic modules and encoded by repeated segments of DNA as explained in example 1.2. These repetitions constitute a problem for the construction of mutations by site-directed mutagenesis. The inventors used the degeneracy of the genetic code to design a mutant allele of the mbp3_{—}16 gene, that was devoid of important repetitions. The mutant allele, mbp3_{—}161 (SEQ ID NO: 5), was synthesized by Genecust (Evry, France) and inserted in the same plasmid vector pQE30 as the parental gene, to give the recombinant plasmid pQEmbp3_{—}161 (SEQ ID NO: 25). Changes of residues were introduced in the DarpMbp3_{—}16 protein at the genetic level, by mutagenesis of either pQEmbp3_{—}16 for A78C and D81C, or pQEmbp3_{—}161 (SEQ ID NO: 6) for the other mutations.

In the present patent application references such as A78C refer to residue 78 of the DarpMbp3_{—}16 protein, which is modified from residue A to C. Following the international one letter amino acid code, hence A=alanine and C=cysteine.

### 1.4 Protein Production, Purification and Characterization

The MalE protein was produced in the cytoplasm of the recombinant strain XL1-Blue(pQEMBP), bt-MalE in strain AVB99(pAT224) and DarpMbp3_{—}16 and its mutant derivatives in XL1-Blue (pQEmbp3_{—}16) or XL1-Blue (pQE3mbp3_{—}161) and their mutant derivates, as described (Binz et al. 2003; Binz et al. 2004). They were purified through their hexahistidine tag by affinity chromatography on a column of fast-flow Ni-NTA resin, as recommended by the manufacturer (Qiagen).

The purification fractions were analyzed by SDS-PAGE, with the NuPAGE Novex system, MES buffer and See blue pre-stained standards (all from Invitrogen). Equal amounts of protein were loaded on the gels after heat denaturation either in the presence or in the absence of 2.5% (v/v, 0.4 M) 2-mercaptoethanol. The gels were stained with Coomassie blue and the protein bands were quantified with the Un-scan-it software (Silk Scientific).

The fractions that were pure after SDS-PAGE in reducing conditions (>98% homogeneous), were pooled and kept at −80° C.

The protein concentrations were measured by absorbance spectrometry, with coefficients of molar extinction, ε₂₈₀(MalE)=66350 M⁻¹ cm⁻¹, ε₂₈₀(DarpMbp3_{—}16)=16960 M⁻¹ cm⁻¹, ε₂₈₀(bt-MaIE)=71850 M⁻¹ cm⁻¹, and ε₂₈₀(DarpOff7)=16960 M⁻¹ cm⁻¹ calculated as described (Pace et al. 1995). Proteins were characterised at 25° C. Aliquots of the wild type DarpOff7(wt) and its mutant derivative DarpOff7(N45C) were analyzed by mass spectrometry after extensive dialysis against 65 mM ammonium bicarbonate and lyophilization, as described (Renard et al. 2002).

All the characterizations of proteins were performed at 25° C.

In addition, those of the cysteine mutants of DarpMbp3_{—}16 were performed in the presence of 5 mM DTT to reduce any intermolecular disulfide bond.

### 1.5 Indirect ELISA

ELISA experiments were performed in buffer M1 and micro-titer plates as described (Harlow and Lane 1988), except that the wells of the plates were washed three times with 0.05% (v/v) Tween 20 in buffer M1 and three times with buffer M1 alone between each step. The wells were coated with 0.5 μg ml⁻¹ DarpOff7 and blocked with 3% BSA (w/v). The immobilized Darpin was incubated with bt-MalE and varying concentrations of potassium iodide KI in 1% BSA for 1 h at 25° C. bt-MalE was omitted in the blank wells. The captured molecules of bt-MalE were revealed with a conjugate between streptavidin and alkaline phosphatase, and p-nitrophenyl phosphate as a substrate (all from Sigma-Aldrich). The absorbance at 405 nm, A₄₀₅, was measured and corrected by subtraction of the blank.

### 1.6 R_{eq} Measurement by Biacore

The Biacore experiments were performed at a flow rate of 25 μL min⁻¹ with streptavidin SA sensorchips (Biacore). A first cell of the sensorchip was used as a reference, i.e. no ligand was immobilized on the corresponding surface. A second cell was loaded with a high density of the bt-MalE protein (>2000 Resonance Units, RU). The DarpMbp3_{—}16 derivatives, at a concentration C=50 nM in buffer M3, were injected for 6 min to monitor association and the buffer alone was injected for 2 min to monitor dissociation.

The chip surface was regenerated between the runs by injection of 10 mM glycine-HCl, pH 3.0, for 24 s. The experimental data were cleaned up with the Scrubber program (Biologic Software) and analyzed with the Biaevaluation 4.1 program (Biacore) to determine R_{eq}, the resonance signal at equilibrium. R_{eq}, is related to the dissociation constant K_{d} by equation (Nieba et al. 1996):

R_{eq}=RₘₐₓC/(C+K_{d}) (1)

Two independent measurements were performed for each DarpMbp3_{—}16 derivative.

### 1.7 Affinity in Solution and Kinetic Measurements by Competition Biacore

The binding reactions (100 μL) were conducted by incubating a fixed concentration of DarpMbp3_{—}16 molecules with variable concentrations of MalE in buffer M2 for >1 hour. The wild type DarpMbp3_{—}16 and its mutant derivatives were used at a concentration of 50 mM, except those carrying mutations T79C, D81C and W90C, which were used at 500 nM to obtain a sufficient signal. It results from the laws of mass action and conservation that:

[P]=0.5{[P]₀−[A]₀−K_{d}+(([P]₀−[A]₀−K_{d})²+4 K_{d}[P]₀)^{1/2}} (2)

where [A]₀ is the total concentration of MalE in the reaction mixture; [P]₀, the total concentration of DarpMbp3_{—}16; and [P], the concentration of free DarpMbp3_{—}16 (Lisova et al. 2007). The association between the reaction mixture at equilibrium and immobilized bt-MaIE was monitored as described below. In these conditions, the initial slope r of the corresponding association curve follows the equation (Nieba et al. 1996):

r=r₀[P]/[P]₀ (3)

where r₀ is the value of r for [A]₀=0. The values of K_{d} and r₀ were determined by fitting equation 3, in which [P] is given by equation 2, to the experimental values of r.

The kinetic measurements were performed at a flow rate of 25 μL min⁻¹ with SA sensor chips. A first cell of the sensor chip was used as a reference, i.e. no ligand was immobilized on the corresponding surface. A second cell was loaded with 500 to 1000 resonance units (RU) of bt-MalE. Solutions of the DarpOff7 derivatives at 8 different concentrations (0.15 to 400 nM) were injected during 8 min to monitor association and then buffer alone during the same time for dissociation. The chip surface was regenerated between the runs by injecting 5 to 10 mM NaOH during 1 min. The signal of the buffer alone was subtracted from the raw signals to obtain the protein signals, and then the protein signal on cell 1 was subtracted from the protein signal on cell 2 to obtain the specific signal of interaction. The kinetic data were cleaned up as above and then the kinetic parameters were calculated by a procedure of global fitting, as implemented in the Bia-evaluation 3.0 software (Biacore). For the wild type DarpOff7 (SEQ ID NO: 28) and its cysteine mutants, the inventors applied a simple kinetic model of Langmuir binding to analyze the data. For the preparations of conjugates, the inventors applied a model with two populations of analytes, whose respective proportions corresponded to the coupling yield y_{c} of the fluorophore.

### 1.8 Fluorophore Coupling

The fluorescent conjugates were synthesised from the cysteine mutants of DarpOff7 essentially as described below.

The cysteine mutants of DarpOff7 were reduced with 5 mM DTT for 30 min at 30° C. with gentle shaking and then transferred into PBS by size exclusion chromatography with a PD10 column (GE Healthcare). The thiol-reactive fluorophore IANBD ester was added in >5:1 molar excess over the Darpin and the coupling reaction was carried out for 2 hours at 30° C. with gentle shaking. The denatured proteins were removed by centrifugation for 30 min at 10000 g, 4° C. The conjugate was separated from the unreacted fluorophore by chromatography on a Ni-NTA column and elution with 100 mM imidazole in buffer H. The coupling yield y_{c}, i.e. the average number of fluorophore molecule coupled to each Darpin molecule, was calculated as described below, with ε₂₈₀(ANBD)=2100 M⁻¹ cm⁻¹, ε₅₀₀(ANBD)=31800 M⁻¹ cm⁻¹, both measured with conjugates between IANBD and 2-mercaptoethanol (Renard et al. 2002).

The fluorescent conjugates were synthesised from the cysteine mutants of DarpMbp3_{—}16 essentially as described below.

The cysteine mutants of DarpMbp3_{—}16 were reduced with 5 mM DTT for 30 min at 30° C. with gentle shaking and then transferred into PBS by size exclusion chromatography with a PD10 column (GE Healthcare). The thiol-reactive fluorophore IANBD ester was added in 10:1 molar excess over the Darpin and the coupling reaction was carried out for 2.5 hours at 30° C. with gentle shaking. The denatured proteins were removed by centrifugation for 30 min at 10000 g, 4° C.

The conjugate was separated from the unreacted fluorophore by chromatography on a Ni-NTA column and elution with 200 mM imidazole in buffer H.

The coupling yield y_{c}, i.e. the average number of fluorophore molecule coupled to each Darpin molecule, was calculated as described below, with ε₂₈₀(ANBD)=2100 M⁻¹ cm⁻¹, ε₅₀₀(ANBD)=31800 M⁻¹ cm⁻¹, both measured with conjugates between IANBD and 2-mercaptoethanol (Renard et al. 2002).

Let P be a protein; B, a mono-conjugate between P and IANBD; Φ, the conjugated form of IANBD; A₂₈₀ and A₅₀₀, the absorbancies of the mixture of P and B that results from the coupling reaction and elimination of the unconjugated fluorophore. Because (i) absorbancies are bilinear functions of molar absorbancies and concentrations, (ii) the molar absorbancies of different chemical groups in a protein molecule are generally additive (Pace et al. 1995), and (iii) proteins generally do not absorb at 500 nm, one can write:

y_{c}=[B]/([B]+[P]) (4)

y_{c}⁻¹=(A₂₈₀/ε₂₈₀(P))(A₅₀₀/ε₅₀₀(Φ))⁻¹−ε₂₈₀(Φ)/ε₂₈₀(P) (5)

where [B] and [P] are concentrations, and ε is a molar absorbance. There is a corrective term in y_{c}⁻¹, which is constant and comes from the contribution of Φ to A₂₈₀.

### 1.9 Fluorescence Measurements and Target Binding

The inventors assumed for the binding and fluorescence experiments at equilibrium that the preparations of conjugates were homogeneous, i.e. that the coupling yield y_{c} was equal to 1. The binding reactions with DarpOff7 conjugates were conducted by incubating 0.3 μM of conjugate with variable concentrations of the MalE antigen in a volume of 1 mL, for 1 hour in the dark with gentle shaking: They were established in buffer L1, or buffer M1, or in a mixture v:(1-v) of calf serum and buffer M1. The conjugate (or biosensor) B and antigen A form a complex B:A according to reaction:

B+A⇄B:A (6)

At equilibrium, the concentration [B:A] of the complex is given by the equation:

[B:A]=0.5{[B]₀+[A]₀+K_{d}−(([B]₀+[A]₀+K_{d})²−4 [B]₀[A]₀)^{1/2}} (7)

where K_{d} is the dissociation constant between A and B, and [A]₀ and [B]₀ are the total concentrations of A and B in the reaction, respectively (Renard et al. 2003).

The fluorescence of the IANBD conjugates was excited at 485 nm (2.5 nm slit width) and its intensity measured between 520 and 550 nm (5 nm slit width) with a FP6300 spectrofluorometer (Jasco).

The signal of MalE alone was measured in an independent experiment and subtracted from the global signal of the binding mixture to give the specific fluorescence intensity F of each conjugate. The intensity F satisfies the following equation:

(F−F₀)/F₀=ΔF/F₀=(ΔF_{∞}/F₀)([B:A]/[B]₀) (8)

where F₀ and F_{∞} are the values of F at zero and saturating concentration of A (Renard et al. 2003). For each conjugate, the inventors first determined the wavelength λₘₐₓ at which the fluorescence intensity of a mixture of conjugate (1 μM) and MalE (10 μM) was maximum and subsequently performed all the measurements of fluorescence at this fixed wavelength. The values of ΔF_{∞}/F₀, [B]₀ and K_{d} could be determined by fitting equation 8, in which [B:A] is given by equation 7, to the experimental values of ΔF/F₀, measured in a titration experiment.

The sensitivity s and relative sensitivity sᵣ of a conjugate are defined by the following equations, for the low values of [A]₀:

ΔF=s[A]₀ (9)

ΔF/F₀=sᵣ[A]₀/[B]₀ (10)

s and sᵣ can be expressed as functions of characteristic parameters of the conjugate:

sᵣ=(ΔF_{∞}/F₀)[B]₀/(K_{d}+[B]₀)) (11)

s=f_{b}sᵣ (12)

where f_{b} is the molar fluorescence of the free conjugate (Renard and Bedouelle 2004). Equation (9) implies that the lower limit of detection δ[A]₀ of the conjugate is linked to the lower limit of measurement of the spectrofluorimeter δF by the proportionality factor s⁻¹.

For the DarpMbp3_{—}16 conjugates, the binding reactions were conducting by incubating 1 μM of conjugate with variable concentrations of the MalE target in a volume of 1 mL, for 30 minutes in the dark with gentle shaking. They were established in buffer M1. The conjugate (or biosensor) B and target A were then considered using the series of equations (6) to (12) detailed above.

### 1.10 Quenching by Potasium Iodide

The experiments of fluorescence quenching by KI were performed at 25° C. in buffer M1, essentially as described above. The Stern-Volmer equation 13 was fitted to the experimental data, where F and F⁰ are the intensities of fluorescence for the DarpOff7 conjugate, with or without quencher, respectively. The Stern-Volmer constant K_{SV} was used as a fitting parameter.

F⁰/F=1+K_{SV}[KI] (13)

### 1.11 Affinity in Solution as Determined by Competition Biacore

The binding reactions (100 μl) were conducting by incubating 50 nM of DarpOff7 with variable concentrations of MalE in buffer M2 or L2 for 1 hour. It results from the laws of mass action and conservation that:

[P]=0.5{[P]₀−[A]₀−K_{d}+(([P]₀−[A]₀−K_{d})²+4 K_{d}[P]₀)^{1/2}} (14)

where [A]₀ is the total concentration of MalE in the reaction mixture; [P]₀, the total concentration of DarpOff7; and [P], the concentration of free DarpOff7 (Lisova et al. 2007). The concentration of free DarpOff7 was measured by Biacore, essentially as described (Nieba et al. 1996). High densities of MalE (>2000 Resonance Units, RU) were immobilized on the surface of a streptavidin SA sensor-chip (Biacore). Each reaction mixture was injected in the sensor chip at a flow rate of 25 μL min⁻¹. The chip surface was regenerated by injecting 10 μL of a Glycine-HCl solution at pH 3.0 (Biacore) between each run. The experimental data were cleaned up with the Scrubber program (Biologic Software) and analyzed with the Bia-evaluation 2.2.4 program (Biacore) to determine the initial slope r of the association curves, which satisfies the equation (Nieba et al. 1996):

r=kₒₙRₘₐₓ[P] (15)

where kₒₙ is the rate constant of association between the free molecules of DarpOff7 and the immobilized molecules of MalE, and Rₘₐₓ is the resonance signal which is obtained with a saturating concentration of DarpOff7. The inventors checked that Rₘₐₓ was not altered by the regeneration of the chip surface and remained constant. Therefore:

r=r₀[P]/[P]₀ (16)

where r₀ is the value of r for [A]₀=0. The values of K_{d} and r₀ were determined by fitting equation (16), in which [P] is given by equation (14), to the experimental values of r.

### 1.12 Data Analysis

The crystal structure of DarpE3_{—}5 (PDB 1MJ0) was analyzed with the What If program (Vriend 1990). In particular, the contacts between the randomized residues of DarpE3_{—}5 were identified with the ANACON routine, using extended Van der Waals radii as described (Rondard and Bedouelle 1998). The fittings of equations to experimental data were performed with the Kaleidagraph software (Synergy Software). The standard errors (SE) on the free energy of dissociation ΔG=−RTlogK_{d} were deduced from the SE values on K_{d} by the equation:

SE(ΔG)=RTSE(K_{d})/K_{d} (17)

The SE value on the variation of interaction energy resulting from a mutation ΔΔG=ΔG(wt)−ΔG(mut) was deduced from the SE values on ΔG by the equation:

[SE(ΔΔG)]²=[SE(ΔG(wt))]²+[SE(ΔG(mut))]² (18)

The residues which are varied in the designed ankyrin repeat domains of DarpE3_{—}5 and DarpMbp3_{—}16 are the same.

As explained above the inventors therefore targeted all the fully randomized positions of DarpMbp3_{—}16 and neglected residues 69 and 102, which are only partially randomized and are located on a different side of the molecule as predicted from the structure of the canonical Darp3_{—}5 (FIG. 9).

### EXAMPLE 2

### Biosensors Based Upon Darpins for which Structural Data is Known—Results Using Biosensors Derived from DarpOff7

### 2.1 Design of the Conjugates

The inventors searched for sites to couple the fluorophore to DarpOff7 that satisfied two principles.

1) The environment of the coupling residue should change between the free and bound states of DarpOff7, so that the environment of the fluorophore would also change between the free and bound states of the conjugate, after coupling.

2) The coupling residue should not be involved in the interaction between DarpOff7 and MalE, so that the fluorophore would not interfere with the interaction between the conjugate and MalE.

The inventors applied these two principles by using the crystal structure of the complex between DarpOff7 and MalE. They identified the residues of DarpOff7 whose solvent accessible surface area (ASA) varied between its free state and its MalE-bound state. They divided this initial set of residues ‘S’ into three subsets. Subset S1 contained the residues of S in direct contact with MalE. Subset S2 contained the residues of S that were in indirect contact with MalE, through a water molecule. Subset S3 contained the residues of S without any contact, either direct of indirect, with MalE (Table 1, FIG. 1).

In FIG. 1 the positions of the coupling sites in the structure of DapOff7. The ankyrin repeats are represented in alternating light grey and dark grey, with the N-cap on top. Residues in direct contact with MalE (subset S1), residues in indirect contact with MalE, through a water molecule (subset S2) and residues whose solvent ASA varies on the binding of MalE and which are in contact with MalE neither directly nor indirectly (subset S3) are listed in table 1 below and have been labelled with their residue numbering FIG. 1. The residues of S2 and S3 were targeted for the coupling of IANBD. DarpOff7 is shown in FIG. 1 from the position of MalE in their complex.

TABLE 1

| | ΔASA | | |
|---|---|---|---|
| Residue | (Å²) | Contact | Set |
| Arg23 | 9.1 | None | S3 |
| Asn45 | 9.0 | None | S3 |
| Thr46 | 23.9 | HOH15 | S2 |
| Thr48 | 18.6 | MalE | S1 |
| Leu53 | 12.2 | None | S3 |
| Tyr56 | 49.8 | MalE | S1 |
| Asp77 | 9.2 | HOH94 | S2 |
| Val78 | 34.2 | MalE | S1 |
| Phe79 | 116.6 | MalE | S1 |
| Tyr81 | 47.0 | MalE | S1 |
| Leu86 | 26.6 | MalE | S1 |
| Tyr89 | 58.7 | MalE | S1 |
| Trp90 | 97.0 | MalE | S1 |
| Asp110 | 10.1 | MalE | S1 |
| Ser111 | 0.8 | None | S3 |
| Asp112 | 17.9 | None | S3 |
| Met114 | 0.7 | HOH192 | S2 |
| Leu119 | 0.2 | None | S3 |
| Lys122 | 1.6 | HOH29, 132 | S2 |
| Trp123 | 64.3 | MalE | S1 |
| Tyr125 | 13.6 | MalE | S1 |

Table 1 shows the analysis of the interface between DarpOff7 and MalE in the crystal structure of their complex. Column 1, residues of DarpOff7 for which ΔASA≠0. Column 2, variation of ASA between the free and MalE-bound states of DarpOff7 for the residues listed in column 1. Column 3, molecules in contact with the residue of column 1. Column 4, sub-set of the residues in column 1: S1, residues in direct contact with MalE; S2, residues in contact with MalE through a water molecule; S3, residues not in contact. The water molecules are numbered according to the PDB file 1SVX. HOH29 and HOH132 belong to a network of six water molecules (HOH20, 110, 29, 132, 147, 171) that are hydrogen-bonded and located in the interface between DarpOff7 and MalE.

The classifications of the residues were identical when the inventors considered the whole residues or only their side-chains. The inventors targeted the coupling of the fluorophore to the residues of subsets S2 and S3, and rejected those of subsets S1 to avoid affecting the binding affinity between DarpOff7 and MalE. However, the inventors also rejected residues Asp77 and Leu119 for the following reasons. Asp77 of DarpOff7 is indirectly hydrogen-bonded to Lys202 of MalE through a water molecule (HOH94) and such indirect hydrogen bonds can be energetically important (England et al. 1997). The variation of ASA for Leu119 on MalE binding is very small, 0.2 Å². The inventors thus selected eight residues of DarpOff7 as potential coupling sites. As a negative control for the design, the inventors chose residue Lys68, which is located on the side of DarpOff7 that is opposite the MalE binding site.

### 2.2 Production and Oligomeric State of the Cysteine Mutants

The eight targeted residues of DarpOff7 and the control residue were changed individually into cysteine by site-directed mutagenesis of the coding gene. The mutant Darpins were produced in the cytoplasm of E. coli at 37° C. and purified through their hexahistidine tag. The yield of purified soluble protein varied between 30 mg/L and 100 mg/L of culture. It varied as much between different mutants as between different batches of the same mutant, and was consistent with that reported previously for the wild type DarpOff7 (SEQ ID NO: 28) (Binz et al. 2004).

The introduction of a cysteine residue could lead to intermolecular disulfide bonds. To characterize the oligomeric state of the DarpOff7 mutants, the inventors analyzed the purified preparations by SDS-PAGE after denaturation in the presence or absence of a reducing agent. In reducing conditions, they observed a single protein species with an apparent molecular mass comprised between 16600 and 16900, and consistent with the theoretical mass of a DarpOff7(wt) monomer, 18272.4. In non-reducing conditions, they observed a second species with an apparent molecular mass comprised between 34100 and 35700, and consistent with the theoretical mass of a dimer, 36542.9. The proportion of monomers in a dimeric state was calculated from the intensities of the protein bands. It varied widely between different mutants, from 3 to 64% (Table 2).

TABLE 2

| | SEQ ID | ASA(S_{γ}) | Dimer | | |
|---|---|---|---|---|---|
| Mutation | NO: | (Å²) | (%) | y_{c} | yₛ |
| R23C | 31 | 17.3 | 69 | 0.98 | 0.60 |
| N45C | 32 | 26.8 | 63 | 0.57 | 0.73 |
| T46C | 33 | 13.4 | 25 | 0.47 | 0.67 |
| L53C | 34 | 3.9 | 8 | 0.99 | 0.67 |
| K68C | 35 | 28.0 | 11 | 1.02 | 0.67 |
| S111C | 36 | 16.0 | 6 | 0.92 | 0.61 |
| D112C | 37 | 19.0 | 7 | 0.76 | 0.67 |
| M114C | 38 | 8.0 | 4 | 0.93 | 0.56 |
| K122C | 39 | 5.2 | 3 | 1.10 | 0.59 |

Table 2 shows the properties of the cysteine mutants of DarpOff7. Column 1, mutation of DarpOff7. Column 3, ASA of the S_{γ} atom, as measured on a three-dimensional model of the DarpOff7 mutant (see Example 1. Materials and Methods). Column 4, proportion of polypeptides in a dimeric state, in a purified preparation of the DarpOff7 mutant. Column 5, number of molecules of fluorophore per molecule of DarpOff7 in a purified preparation of the conjugate (coupling yield y_{c}). Column 6, yield of synthesis (yₛ) for the preparation of a conjugate from a DarpOff7 mutant.

### 2.3 Conjugation and its Yield

The inventors submitted the purified preparations of the DarpOff7 mutants to a reaction of reduction before coupling with IANBD, to break open potential intermolecular disulfide bonds and ensure that the mutant cysteine would be in a reactive state.

The products of the coupling reaction were separated from the unreacted fluorophore by chromatography on a nickel ion column. The coupling yield y_{c} was calculated from the absorbance spectra of the purified reaction product (see Example 1. Materials and Methods). It was found to be very reproducible, close to 100% for six of the nine DarpOff7 mutants, and lower for the mutants at positions Asp112 (75%), Asn45 (57%) and Thr46 (47%). The synthesis yield yₛ of the coupling procedure, i.e. the proportion of protein molecules that survived the procedure, was close for all the DarpOff7 mutants, 64±5% (mean±SE, Table 2).

The inventors analyzed the cause for the low yield of coupling in position Asn45, so as to have more homogeneous preparations of conjugates. The inventors have found that the low yield of coupling for DarpOff7(N45C) did not result from a low accessibility of the mutant cysteine to the solvent, since this mutant derivative of DarpOff7 could form an intermolecular disulfide bond efficiently. It also did not result from an irreversible modification of the mutant cysteine since an analysis of a purified preparation of DarpOff7(N45C) by mass spectrometry showed that it contained only two protein species, with molecular masses that were equal to 18275.8±1.6 and 36548.7±2.1 and were close to the theoretical masses of the monomeric and dimeric states of DarpOff7(N45C), 18272.4 and 36542.9 respectively. The low yield did not result from an oxidized state of the mutant cysteine because the inventors performed a reducing treatment either before or during the reaction of coupling, with different reducing agents (TCEP or DTT) and at variable concentrations of these agents (from 0.1 to 5 mM), without any change. Moreover, the inventors checked by SDS-PAGE that the protein was in a monomeric state immediately after this treatment. Finally, the low yield did not result from slow coupling kinetics because the observed yield was not changed by an increase in temperature (from 30° C. to 40° C.) or an increase in the duration of the reaction (from 30 min to overnight). Therefore, the inventors are unable to explain the differences in the yields of coupling for DarpOff7(N45C) with the other conjugates.

### 2.4 Fluorescence Properties of the Conjugates

The fluorescence of the conjugates was excited at 485 nm and recorded at 535 nm. The inventors tested the responsiveness of the DarpOff7 conjugates to the binding of their MalE antigen by measuring the relative variation ΔF/F₀=(F−F₀)/F₀ in their fluorescence intensity F between their MalE-bound and free states.

In a first test, the inventors used a concentration of MalE equal to 2.6 μM, i.e. about 9 times the concentration of conjugate (0.3 μM) and 230 times the value of the dissociation constant K_{d} (11 nM) between DarpOff7(wt) and MalE. All the conjugates that the inventors constructed, responded to the binding of MalE, except the DarpOff7(K68ANBD) control. The value of ΔF_{26 μM}/F₀ was between 0.9 and 14.6 for the eight responsive conjugates when the assay was done in the low salt buffer L1 (Table 3).

TABLE 3

| | Buff- | f_{b} | | | K_{d} |
|---|---|---|---|---|---|
| Residue | er | (FU μM⁻¹) | ΔF_{2.6μM}/F₀ | ΔF_{∞}/F₀ | (nM) |
| Arg23 | L1 | 341 ± 2 | 0.96 ± 0.03 | 0.96 ± 0.01 | 26 ± 4 |
| Asn45 | L1 | 232 ± 6 | 14.1 ± 0.4 | 14.00 ± 0.07 | 13 ± 2 |
| Thr46 | L1 | 166 ± 2 | 14.5 ± 0.2 | 18.0 ± 0.2 | 546 ± 40 |
| Leu53 | L1 | 276 ± 2 | 2.61 ± 0.05 | 2.91 ± 0.06 | 271 ± 36 |
| Ser111 | L1 | 271 ± 1 | 0.74 ± 0.01 | 0.73 ± 0.01 | 10 ± 3 |
| Asp112 | L1 | 197 ± 2 | 2.09 ± 0.03 | 2.17 ± 0.04 | 121 ± 19 |
| Met114 | L1 | 56 ± 5 | 8.1 ± 0.9 | 8.9 ± 0.2 | 211 ± 30 |
| Lys122 | L1 | 47 ± 1 | 2.21 ± 0.04 | 122 ± 13 | (4.7 ± 0.3) × |
| | | | | | 10⁶ |
| Arg23 | M1 | 314 ± 2 | nd | 0.93 ± 0.01 | 18 ± 5 |
| Asn45 | M1 | 266 ± 3 | nd | 8.25 ± 0.06 | 8 ± 2 |
| Thr46 | M1 | 323 ± 4 | nd | 7.92 ± 0.08 | 255 ± 18 |
| Leu53 | M1 | 259 ± 3 | nd | 2.61 ± 0.03 | 104 ± 10 |
| Asn45 | Se- | 452 ± 4 | nd | 2.11 ± 0.01 | 18 ± 2 |
| | rum | | | | |

Table 3 shows the properties of DarpOff7 conjugates, as derived from fluorescence experiments. Column 1, residue with which the fluorophore was coupled. Column 3, molar fluorescence f_{b} of the free conjugate. The total concentration of conjugate was equal to 0.3 y_{c} μM, where the coupling yield y_{c} is given in Table 2. The entries for f_{b} and ΔF_{2.6 μM}/F₀ give the mean value and associated standard error (SE) in at least two experiments. The entries for ΔF_{∞}/F₀ and K_{d} give the value and associated SE in the fitting of Equation 8 to the data points in the titration experiments. The Pearson parameter in these fittings was R>0.996. The K_{d} value for DarpOff7(wt) was equal to 11±1 nM in buffer L2 and 5.6±0.8 nM in buffer M2, as measured by competition Biacore. Serum, 90% calf serum; nd, not determined. The SE value on ΔF_{2.6 μM}/F₀ was calculated through the equation [SE(ΔF_{2.6} μM/F₀)]²=(F_{2.6 μM}/F₀)²{[SE(F_{2.6 μM}/F_{2.6 μM}]²+[SE(F₀)/F₀]²}.

The values of ΔF_{2.6 μM}/F₀ varied between conjugates. These variations could come either from different interactions between the fluorescent group and MalE, or from different affinities between the conjugates and MalE. To distinguish between these mechanisms and characterize the properties of the eight conjugates in more details, they determined the relation between the intensity of fluorescence and concentration of MalE for each conjugate by titration experiments in buffer L1 (FIG. 2).

In FIG. 2 the Titration of DarpOff7 conjugates by MalE, monitored by fluorescence. The experiments were performed at 25° C. in buffer M1. The total concentration in DarpOff7, measured by A₂₈₀, was equal to 0.3 μM. The total concentration in the MalE protein is given along the x axis. The continuous curves correspond to the fitting of Equation 5 to the experimental values of ΔF/F₀ (see Materials and Methods for details). (Δ) position Arg23; () Asn45; (◯) Thr46; (▴) Leu53; (♦) Lys68.

The theoretical Equation 8, linking ΔF/F₀ and the concentration of antigen, was fitted to the experimental data with the concentration of functional conjugate, ΔF_{∞}/F₀ and K_{d} as fitting parameters (Table 3). The values of ΔF_{∞}/F₀ and ΔF_{2.6 μM}/F₀ were close for seven of the eight conjugates but differed by 55-fold for DarpOff7(K122ANBD). The values of K_{d} differed widely between conjugates and were comprised between 10 nM and 4.7 mM. These values were several fold lower than 2.6 μM for the seven conjugates above and therefore these conjugates were saturated by MalE at a concentration of 2.6 μM. In contrast, K_{d} was 1800-fold higher than 2.6 μM for DarpOff7(K122ANBD). Therefore this conjugate was not saturated by MalE at 2.6 μM, which explained the large difference between its values of ΔF_{∞}/F₀ and ΔF_{2.6 μM}/F₀ (2.2 and 121 respectively). Note that the high value of ΔF_{∞}/F₀ for DarpOff7(K122ANBD) was obtained through a long range extrapolation and might be an overestimate.

### 2.5 Fluorescence and Salt Effects

The quantum yield of fluorophores and the electrostatic interactions between molecules can be salt sensitive. The salt concentration of the buffer could therefore affect the response of the DarpOff7 conjugates at the levels of both their fluorescent group and interaction with MalE. To test these assumptions, the inventors compared the fluorescence properties of four conjugates, at positions Arg23, Asn45, Thr46 and Leu53, by experiments of titration in the low salt buffer L1 and medium salt buffer M1 (FIG. 3).

In FIG. 3 the selectivity and specificity of the fluorescence signal for the DarpOff7(N45ANBD) conjugate is measured in varying conditions. The experimental conditions were as described in FIG. 2, except for the buffers. The total concentration in antigen, MalE or BSA, is given along the x axis. () MalE in buffer L1; (◯) MalE in buffer M1; (♦) MalE in 90% serum; (⋄) BSA in buffer M1.

The inventors also compared the properties of interaction between the parental DarpOff7(wt) and MalE in these two buffers by experiments of competition Biacore (Example 1. Materials and methods).

The inventors found that the value of K_{d} for DarpOff7(wt) was slightly higher in buffer L1 than in buffer M1, 11±1 nM versus 5.6±0.8 nM, as measured by competition Biacore. The inventors observed the same trend for the K_{d}s of four conjugates as measured by titration experiments, e. g. the value of K_{d} for DarpOff7(T46ANBD) was higher by 2-fold in buffer L1 (Table 3). These results suggested that the NaCl ions screened unfavorable electrostatic interactions. The values of F₀ and ΔF_{∞}/F₀ were very close in buffers L1 and M1 for the conjugates at positions Arg23 and Leu53. In contrast, the values of F₀ were lower in buffer L1 than in buffer M1 for the conjugates at positions Asn45 and Thr46, and consequently the values of ΔF_{∞}/F₀ were higher, up to 2-fold (Table 3). Thus, lower salt concentrations could increase both K_{d} and ΔF_{∞}/F₀ for some conjugates.

### 2.6 Selectivity and Specificity

The selectivity of a biosensor refers to the extent to which it can recognize a particular analyte in a complex mixture without interference from other components in the mixture (Vessman et al. 2001). The inventors tried to characterize the selectivity of the DarpOff7(N45ANBD) conjugate by performing experiments of titration by the MalE antigen in a complex medium like serum and by comparing these experiments with those performed in the medium salt buffer M1 (FIG. 3). The inventors found that the value of ΔF_{∞}/F₀ for DarpOff7(N45ANBD) was high in serum, 2.11±0.01, and that this conjugate recognized MalE with close values of K_{d} in serum and buffer M1 (Table 3). Thus, DarpOff7(N45ANBD) recognized MalE selectively in serum. However, the value of F₀, the fluorescence intensity of the free conjugate, was 1.7 fold higher and the value of ΔF_{∞}/F₀ was 3.9 fold lower in serum than in buffer M1. Thus, some components of the serum interfered with the fluorescence properties of DarpOff7(N45ANBD) as measured by the experimental setting (see below).

The experiments to determine the recognition of MalE by DarpOff7(N45ANBD) in serum and buffer M1 showed that this recognition was selective. To further establish the specificity of the recognition, the inventors titrated DarpOff7(R23ANBD) and DarpOff7(N45ANBD) with bovine serum albumin (BSA) and hen egg white lysozyme in buffer M1. The inventors found that the values of ΔF_{2.6 μM}/F₀ were much lower for the non-cognate proteins than for MalE, e. g. 40-fold lower for BSA (FIG. 3). Therefore, the variation of the ΔF/F₀ signal was indeed specific of MalE, the cognate antigen.

### 2.7 Binding Parameters

The experimental conditions of the titration experiments were not appropriate to evaluate nano-molar values of K_{d} precisely since the concentration of conjugate was micro-molar. The inventors measured the kinetic parameters of interaction between DarpOff7(wt), four of its cysteine mutants, and the four corresponding conjugates on the one hand, and MalE on the other hand by Biacore to obtain more precise values and better understand the mechanisms of variation in these parameters. The kinetics were performed in the presence of DTT for the cysteine mutants and the corresponding controls to prevent their dimerization. The inventors analyzed the kinetic data with the model of a simple bi-molecular reaction (one analyte and one ligand) for DarpOff7(wt) and its cysteine mutants, and calculated the corresponding dissociation constant from the rate constants, i.e. K_{d}′=k_{off}/kₒₙ.

The inventors used the model of a three-molecular reaction (two analytes and one ligand) for the conjugates to take the incomplete coupling of some preparations into account (Table 2). The results of the kinetic experiments are reported in Table 4.

TABLE 4

| | | | kₒₙ₁ | k_{off1} | K_{d1}′ | kₒₙ₂ | k_{off2} | K_{d2}′ |
|---|---|---|---|---|---|---|---|---|
| Derivative | Buffer | Model | (10⁵ M⁻¹ s⁻¹) | (10⁻³s⁻¹) | (nM) | (10⁵ M⁻¹ s⁻¹) | (10⁻³ s⁻¹) | (nM) |
| WT | M2 | LB | 6.6 | 5.1 | 7.7 | Na | na | na |
| WT | L2 | LB | 2.5 | 6.2 | 25.1 | Na | na | na |
| WT | M3 | LB | 5.5 | 6.1 | 11.1 | Na | na | na |
| R23C | M3 | LB | 4.1 | 5.0 | 12.4 | Na | na | na |
| N45C | M3 | LB | 2.9 | 7.4 | 25.7 | Na | na | na |
| T46C | M3 | LB | 1.7 | 4.1 | 24.7 | Na | na | na |
| L53C | M3 | LB | 3.7 | 6.9 | 18.8 | Na | na | na |
| R23ANBD | M2 | HA | 1.8 | 11.7 | 63.2 | 4.3 | 2.7 | 6.2 |
| N45ANBD | M2 | HA | 2.1 | 1.8 | 8.9 | 2.4 | 11.3 | 46 |
| T46ANBD | M2 | HA | 0.64 | 26.1 | 408 | 0.18 | 1.8 | 98 |
| L53ANBD | M2 | HA | 2.0 | 60 | 326 | 5.8 | 4.0 | 6.8 |

Table 4 shows the binding parameters of DarpOff7 and derivatives, as determined by Biacore experiments. The Bt-MalE antigen was immobilized on streptavidin SA sensorchips. The association and dissociation rate constants, kₒₙ and k_{off}, were determined at 25° C. and used to calculate K_{d}′=k_{off/k}ₒₙ (Materials and Methods). The inventors applied a simple kinetic model of Langmuir binding (LB) for DarpOff7(wt) and its cysteine mutants. The inventors applied a model with two populations of analytes (heterogeneous analyte HA) for the preparations of conjugates to take incomplete coupling into account. na, not applicable.

They found that the value of K_{d}, measured at equilibrium in solution by competition Biacore (see Example 1 above), and the value of K_{d}′, deduced from kinetic experiments at the interface between a liquid and a solid phase, were close for DarpOff7(wt) in medium salt buffer (5.6±0.8 nM versus 7.7 nM). The value of K_{d}′ for DarpOff7(wt) was higher in low salt buffer than in medium salt buffer. This variation of K_{d}′ with the concentration in salt was consistent with that of K_{d}, although larger (3.5-fold versus 2-fold).

It was mainly due to an increase of kₒₙ with the concentration in salt and therefore consistent with a long range effect, e.g. the screening of unfavorable electrostatic interactions between DarpOff7(wt) and MalE by the salt. The other kinetics were performed in medium salt buffer. The mutation of residues into Cys had little effect on the values of k_{off}, kₒₙ and K_{d}′. The effects were the most important for mutations N45C and T46C (2.2 fold) and mainly due to a slower kₒₙ.

For the conjugates, the value of K_{d1}′ measured by Biacore was consistent with the value of K_{d} measured by fluorescence. The value of K_{d2}′ was close to that of the corresponding cysteine mutant, except for the preparation of DarpOff7(T46ANBD) for which it was 5.5 fold higher. These kinetic experiments were performed in the absence of a reducing agent, therefore the non-coupled molecules of DarpOff7(T46ANBD) could be in a dimeric state and thus altered in their ability to bind MalE. The value of K_{d1}′ for DarpOff7(N45ANBD) was close to the value of K_{d}′ for DarpOff7(wt); it was 8 fold higher for DarpOff7(R23ANBD) and about 50 fold higher for DarpOff7(T46ANBD) and DarpOff7(L53ANBD). The increase in the K_{d1}′ value of the conjugates relative to the K_{d}′ value of the parental DarpOff7(wt) resulted from variations in both k_{off} and kₒₙ, and the variation of this latter parameter could be important, 10 fold for DarpOff7(T46ANBD).

A comparison between the values of K_{d}′ for the cysteine mutants and K_{d} or K_{d1}′ for the conjugates showed that the variations in affinity were mainly due to the coupling of the fluorophore and not to the mutation into Cys. The values of K_{d}, determined by titration experiments, were comprised between the corresponding values of K_{d1}′ and K_{d2}′, determined by Biacore. This comparison was consistent with K_{d} being an apparent dissociation constant and describing a mixture of conjugated and unconjugated molecules. However, one should keep in mind that dissociation constants in solution and at the interface between solid and liquid phases are not generally equal (Rich and Myszka 2005).

### 2.8 Mechanism of Fluorescence Variation

All eight conjugates that the inventors designed as potential biosensors, were sensitive to the binding of MalE, with ΔF_{∞}/F₀>0.73. To test whether these variations of fluorescence resulted from the proximity between the coupling site of the fluorophore and the binding site of MalE, as assumed in the inventors design scheme, they constructed the negative control DarpOff7(K68ANBD) conjugate. Residue Lys68 is located on the side of DarpOff7 that is opposite to the binding site of MalE. They did not observe any variation of fluorescence for DarpOff7(K68ANBD) on the binding of MalE. This observation suggests that the fluorophore had to be in the neighborhood of the Darpin binding site for the fluorescence to vary.

The inventors used potassium iodide (KI) to explore the physicochemical mechanism by which the fluorescence intensity of the conjugates varied on antigen binding. First, the inventors checked by an indirect ELISA that KI, up to 250 mM, did not affect the interaction between the parental DarpOff7(wt) and MalE (Example 1 Materials and methods). They found that the fluorescence of the DarpOff7(N45ANBD) conjugate was quenched by KI, both in its free and MalE-bound states. The quenching varied linearly with the concentration of KI (FIG. 4).

In FIG. 4 the quenching of the DarpOff7(N45ANBD) fluorescence by KI. F and F⁰, fluorescence of the conjugate at 25° C. in buffer M1, with and without quencher respectively. () Conjugate (1 μM) in the absence of the MalE antigen; (◯) conjugate (0.3 μM) in the presence of a saturating concentration of MalE (1.8 μM). The continuous curves were obtained by fitting Equation 13 to the experimental data.

This law of variation indicated that the molecules of fluorophore constituted a homogeneous population and were identically exposed to KI (Lakowicz 1999). It confirmed that the fluorescent group was specifically coupled to the mutant cysteine. The Stern-Volmer constant was higher for the free conjugate than for its complex with the antigen: K_{SV}=2.92±0.06 M⁻¹ versus 1.06±0.03 M⁻¹ (SE in the curve fits of FIG. 4). These values indicated a lower accessibility of the fluorophore to KI in the bound state of the conjugate than in its free state. They showed that the fluorescence increase was due to a shielding of the fluorescent group from the solvent by the binding of the antigen, as previously observed for other conjugates with IANBD (Renard et al. 2003). Thus the mechanism of fluorescence variation was general and consistent with the rules of design.

### 2.9 Mechanism of Fluorescence Variation in Serum

The profiles of titration of DarpOff7(N45ANBD) by MalE were different in calf serum and in a defined buffer (FIG. 3). In particular, the inventors observed that the value of F_{∞} was lower and that of F₀ higher in serum. To better understand these differences, the inventors measured the variations of the F₀ and F_{2.6 μM} parameters as functions of the concentration in serum (FIG. 5).

In FIG. 5 the effect of the concentration in serum on the fluorescence signals for the DarpOff7(N45ANBD) conjugate. The experiments were performed in a mixture (v: 1- v) of serum and buffer M1. The total concentration of MalE was equal to 2.6 μM and thus saturating. The other experimental conditions were as described in FIG. 2. (◯) F₀; () F_{2.6 μM}; FU, arbitrary units of fluorescence. The continuous curves were obtained by fitting a linear model of attenuation to the experimental values of F_{2.6 μM}, and a mixed model of association and linear attenuation to the values of F₀.

The inventors observed that the value of F_{2.6 μM} for DarpOff7(N45ANBD) decreased linearly with the concentration in serum. As expected, the absorbance of the serum alone increased linearly with its concentration, in agreement with the Beer-Lambert law, at both 485 nm and 535 nm, which were the wavelengths of fluorescence excitation and emission in the experiments. Therefore, the absorption of the excitation and emission lights by serum could account for the variation of F_{2.6 μM}. Surprisingly, F₀ increased with the concentration in serum, up to 40% (v/v) of serum and then decreased slowly. The initial increase could result from the interaction between the DarpOff7 conjugate and molecules of the serum and the subsequent decrease from the absorbance of the serum, as observed for F_{2.6 μM}.

### 2.10 Rules of Design and Their Efficiency

The inventors have developed and validated a method to choose coupling sites for fluorophores in a Darpin and transform it into a reagentless fluorescent biosensor. The method is based on the crystallographic coordinates of the complex between the Darpin and its antigen, and it does not involve any knowledge on their energetic interface. Two criteria were applied: (1) the solvent ASA (accessible surface area) of the target residue should vary between the free and bound states of the Darpin; (2) the target residue should not be in contact with the antigen.

The first rule was based on the assumption that the fluorescence variation of the conjugate upon antigen binding is due to a change in the environment of the fluorescent group. The second rule aimed at avoiding residues that contribute to the energy of interaction between the Darpin and its antigen.

The inventors applied this method to the complex between DarpOff7 and its target MalE, and thus selected eight coupling residues in DarpOff7. Each of them gave a conjugate that could detect the binding of MalE with a value ΔF_{∞}/F₀>0.73. Three conjugates had affinities close to that of DarpOff7(wt) (ΔΔG≦0.5 kcal mol⁻¹). The most promising conjugate, DarpOff7(N45ANBD), had a value ΔF_{∞}/F₀=14.0±0.1 and an affinity nearly identical to that of DarpOff7(wt) (ΔΔG=0.1±0.01 kcal mol⁻¹). Experiments of fluorescence quenching by KI with the DarpOff7(N45ANBD) conjugate showed that the mechanism of fluorescence variation was consistent with the rules of design and general mechanisms of fluorescence variability.

The conjugates that were constructed from the three residues that were in indirect contact with the antigen (Thr46, Met114 and Lys122, belonging to subset S2), had the lowest values of F₀ and the highest values of ΔF_{∞}/F₀. Residues Thr46 and Met114 make indirect contacts with MalE through a single and isolated water molecule (HOH15 and HOH192 respectively). Lys122 makes indirect contacts with MalE through two water molecules (HOH29 and HOH132) which in turn belong to a network of six water molecules, linked by hydrogen bonds. The corresponding conjugate DarpOff7 (K122ANBD) had an exceptionally high value of ΔF_{∞}/F₀. The low F₀ values suggested that the fluorescent group was highly exposed to the solvent in the free state of these conjugates. The positions of the water molecules and high ΔF_{∞}/F₀ values suggested that the fluorescent group displaced water molecules in the interface between DarpOff7 and MalE in the bound state of these conjugates, and was at least partially buried in this interface. Consistently, the affinities between the three corresponding conjugates and MalE were also much decreased.

Residue Asn45, which belonged to the S3 subset, is adjacent to residue Thr46, which belonged to the S2 subset. It is farther from the interface between DarpOff7 and MalE than Thr46. The corresponding conjugate DarpOff7(N45ANBD) had a very high value ΔF_{∞}/F₀=14.0±0.1 and an unchanged affinity relative to DarpOff7(wt). Its fluorescent group might have replaced HOH15, as the DarpOff7(T46ANBD) one, but without inserting itself as much in the interface. The high values of ΔF_{∞}/F₀ that the inventors obtained for some conjugates, showed that the use of the IANBD ester as a fluorophore did not limit the extent of the fluorescence response a priori.

### 2.11 Production and Dimerization of the Cys Mutants

Some mutant derivatives of DarpOff7, carrying a Cys mutation, formed covalent homodimers through an intermolecular disulfide bond. The relative proportions of monomers and homodimers in the protein preparations varied with the position of the mutation. The inventors assumed that this dimerization occurred during the cellular extraction and purification of proteins since disulfide bonds do not form in the reducing medium of the cytoplasm. The inventors modeled the three-dimensional structure of the mutant DarpOff7 molecules and calculated the solvent ASA of the mutant cysteines (Table 2). As expected, a low accessibility of the S_{γ} atom to the solvent disfavored the formation of a disulfide bond (e.g. at positions Leu53, Met114 and Lys122) whereas a high accessibility favored it (e.g. at positions Arg23 and Asn45) but was not sufficient (e.g. at positions Lys68, Asp112C and Ser111).

Likely, the geometrical relationships that are necessary to form a disulfide bond, were not satisfied for these three last mutations (Sowdhamini et al. 1989). Nevertheless, the results demonstrated the possibility of linking two Darpin molecules together through a disulfide bound, which could be used to design bivalent or bifunctional Darpin dimers.

### 2.12 Impact of the Fluorescent Group on Antigen Binding

The ΔF_{∞}/F₀ and K_{d} parameters were obtained by fitting Equation 8 to titration data. This equation describes the association of homogeneous preparations of protein and antigen. However, MalE was in contact with different DarpOff7 species for the conjugates with a coupling yield <100%, i.e. the conjugated species, the cysteine mutant in a monomeric unconjugated form and the mutant in a homodimeric form. The value of ΔF_{∞}/F₀, which is a relative, dimensionless parameter, was not affected by the coupling yield, provided that the coupling was homogeneous.

The inventors have shown that such was the case for the DarpOff7(N45ANBD) conjugate in the experiments of fluorescence quenching by KI (see Results). Moreover, the real value of K_{d} for the interaction between the conjugated species and MalE was necessarily lower than the apparent value of K_{d} that they obtained with Equation 8 since the concentration of antigen that was available to the conjugate, was lower than or equal to the total concentration. Therefore, the values of ΔF_{∞}/F₀ that they report in Table 3, represent the correct value despite the approximation and the values of K_{d} are over-estimation, i.e. the real affinities of the conjugates for MalE were higher or equal to the apparent affinities that they found. The value of K_{d} for the DarpOff1(N45ANBD) conjugate in the titration experiments was compatible with its K_{d1}′ value in the Biacore experiments. Moreover, the K_{d2}′ value for this conjugate was consistent with the K_{D}′ value for the unconjugated cysteine mutant in the Biacore experiments (Tables 3 and 4). These comparisons indicated that the parameters that the inventors determined to characterize this conjugate, were reliable.

The inventors have shown that the K_{d}′ value, measured for four of the cysteine mutants, were only 1.1 to 2.3 fold higher than the K_{d}′ value for DarpOff7(wt). Therefore, the variations of K_{d}′ that they observed for the conjugates at positions 23, 46 and 53, were mainly due to the presence of the fluorescent group, which affected the interaction between DarpOff7 and MalE.

### 2.13 Classification of the Conjugates

The conjugates of DarpOff7 had a wide diversity of values for ΔF_{∞}/F₀ and K_{d}. The inventors classified them according to their sensitivity, a parameter which is used to characterize any measuring instrument. This sensitivity can take two forms for a RF biosensor, a relative sensitivity sᵣ and an absolute sensitivity s.

The relative sensitivity sᵣ relates the relative variation of the fluorescence signal ΔF/F₀ to the relative concentration of antigen [A]₀/[B]₀ for the low values, where [A]₀ and [B]₀ are the total concentrations of antigen and conjugate, respectively, in the measuring reaction (Equation 10 in Example 1. Materials and Methods). sᵣ is an intrinsic dimensionless parameter. Its value does not depend on the spectrofluorometer or its set up, and should remain constant between experiments, instruments and laboratories. The value of sᵣ depends on the values of [B]₀ and K_{d} according to a saturation law and its maximal value is equal to ΔF_{∞}/F₀ (Equation 11).

The absolute sensitivity s relates ΔF and [A]₀ for the low values and is equal to f_{b}sᵣ, where f_{b} is the molar fluorescence of the free conjugate (Equations 9 and 12). The s⁻¹ parameter relates the lower limit of detection δ[A]₀ for the conjugate to the lower limit of measurement δF for the spectrofluorometer.

The inventors calculated the variations of sᵣ and s⁻¹ for each conjugate as a function of [B]₀ in the low salt buffer L1 (FIGS. 6 and 7).

In FIG. 6 the ranking of the DarpOff7 conjugates according to their relative sensitivities sᵣ at 25° C. in buffer L1. The sᵣ parameter relates the relative variation of fluorescence intensity ΔF/F₀ and the relative concentration of antigen [A]₀/[B]₀ for the low values of [A]₀, where [A]₀ and [B]₀ are the total concentration of antigen and conjugate in the binding reaction, respectively (Equations 10 and 11). (▪) position Arg23; () Asn45; (◯) Thr46; (▴) Leu53; (□) Ser111; (Δ) Asp112; (▾) Met114; (♦) Lys122.

In FIG. 7 the Ranking of the DarpOff7 conjugates according to their lower limit of detection at 25° C. in buffer L1. The s⁻¹ parameter gives the lower concentration of antigen [A]₀ that can be detected by a conjugate when the lower variation of fluorescence intensity that can be detected by the spectrofluorometer, is equal to 1 FU. (▪) position Arg23; () Asn45; (◯) Thr46; (▴) Leu53; (□) Ser111; (Δ) Asp 112; (▾) Met 114.

These variations showed that the classification of the conjugates varied as a function of [B]₀. For sᵣ and with [B]₀=0.3 μM, i.e. the concentration at which the inventors performed there experiments, the coupling positions ranked in the following order: Asn45>Thr46>Met114>Leu53≈Asp112>Arg23>Ser111. For s⁻¹ and [B]₀=0.3 μM, the coupling positions ranked in the following order: Asn45<Thr46<Leu53<Arg23≈Asp112≈Met114<Ser111. DarpOff7(N45ANBD) at 0.3 μM had a value s⁻¹=0.32 nM FU⁻¹ and therefore a lower limit of detection δ[A]₀=0.32 nM in the experiments since the Perkin-Elmer SF5B spectrofluorometer could detect a variation of fluorescence δF=1 FU.

### 2.14 Conclusions

The inventors have developed a method to construct reagentless fluorescent (RF) biosensors from Darpins when the crystal structure of the complex with the antigen is available. This method could be applied to any antigen binding protein in the same conditions. The inventors have validated the method by constructing eight conjugates between the IANBD fluorophore and DarpOff7, a Darpin that is directed against the MalE protein from E. coli. The inventors ranked the conjugates according to their relative sensitivity sᵣ and their lower limit of detection (proportional to s⁻¹) and showed that this ranking depended on the concentration in conjugate. One of the conjugates had values sᵣ>6 and s⁻¹<0.7 nM for a concentration of the conjugate equal to 10 nM, and sᵣ>12 and s⁻¹<0.35 nM for a concentration of the conjugate equal to 100 nM. It could function in a complex mixture like serum and the mechanism of its fluorescence variation was general. An analysis of the results on DarpOff7 allowed the inventors to propose a method to construct RF biosensors from Darpins whose structure is unknown. The yields of production of DarpOff7 and its cysteine mutants, and the yields of synthesis of the conjugates with the IANBD fluorophore were much higher than those for scFv fragments of antibodies. The sensitivities of the conjugates from DarpOff7 were generally several fold higher than those from scFv fragments. Therefore, the Darpins, which are very stable proteins, constitute a promising alternative to antibody fragments for the construction and the multiple applications of reagentless fluorescent biosensors, directed against any protein antigen.

### EXAMPLE 3

### Biosensors Based Upon Darpins for which No Structural Data is Known—Results Using Biosensors Derived from DarpMbp3_{—}16

### 3.1 Rationale for the Choice of Coupling Sites

The inventors first considered a set R of the residue in positions that are randomized in the combinatorial library of Darpins. They call these positions “randomized positions” of the Darpin under consideration for simplicity. Their side-chains are not essential for the folding of Darpins and exposed to the solvent by design of the library. Set R can be divided in three disjoint subsets R₁ to R₃. R₁ is the set of the positions that have an energetic importance for the interaction between the Darpin and its target. R₂ is the set of the positions that are not important for the interaction but are adjacent in the sequence or structure of the Darpin to positions of R₁. R₃ is the set of the positions that are neither energetically important nor adjacent to positions of R₁. The inventors assumed that the residues at the R₁ positions are generally in contact with the target in the complex between a Darpin and its target, and considered that the R₁ positions should be avoided for the coupling of a fluorophore because the fluorescent group would interfere with the binding between the Darpin conjugate and its target (see above).

The inventors considered that the positions of R₂ are potential targets for the coupling of a fluorophore to a Darpin because they are not involved in the binding of its target and they have a good probability of being in the neighborhood of the target in their complex. Finally the positions of R₃ are less likely than those of R₂ to be in the neighborhood of the target.

Here, the inventors considered that a residue R₂ is adjacent to another residue R_{I} along the sequence of a Darpin if R₂ is in position n−1 or n+1 relative to position n of R₁. Residues R₁ and R₂ are adjacent in the structure of a Darpin if they are in Van der Waals contact. Briefly, the inventors used published Van der Waals radii (Gelin and Karplus, 1979) and considered that two atoms are in Van der Waals contact if their distance is lower or equal to 1.11 times the sum of their radii, as recommended (Sheriff et al., 1987; Sheriff, 1993). In the present Patent Application a Darpin residue is located in the neighborhood of the corresponding target if the binding of the target modifies its solvent accessible surface area.

To test the above rationale, the inventors constructed conjugates between DarpMbp3_{—}16, a Darpin which has two designed ankyrin repeats and is directed against the MalE protein from E. coli, and IANBD, a fluorophore which is sensitive to its environment.

The inventors targeted all the fully randomized positions of DarpMbp3_{—}16, namely positions 43, 45, 46, 48, 56, 57, 76, 78, 79, 81, 89, 90 which correspond to Xaa residues in the designed ankyrin repeat consensus (SEQ ID NO: 7) and did not modify positions 69, 102 and 135 which correspond to residues which are only partially randomized in the designed ankyrin repeat consensus (SEQ ID NO: 7). The inventors thus introduced Cys residues in 12 randomized positions of DarpMbp3_{—}16 (see Table 5) and characterized the properties of the mutant derivatives and corresponding conjugates. The conjugates were ranked according to their relative sensitivities and the best five were studied in more detail.

### 3.2 Production of the Conjugates.

The residues at the twelve fully randomized positions of DarpMbp3_{—}16 were changed individually into cysteine by site-directed mutagenesis of the coding gene. The mutant Darpins were produced in the cytoplasm of E. coli at 37° C. and purified through their hexahistidine tag. The yield of purified soluble protein varied between 30 mg/L and 100 mg/L of culture. It varied as much between different mutants as between different batches of the same mutant, and was consistent with that reported previously for other Darpins (Kohl et al., 2003).

The twelve altered DarpMbp3_{—}16 cysteine mutants are listed in Table 5, first column.

TABLE 5

| DarpMbp3_16 | R_{eq} | K_{d} | ΔΔG | |
|---|---|---|---|---|
| Mutation | (RU) | (nM) | (kcal mol⁻¹) | y_{c} |
| WT | 400 ± 2 | 43.2 ± 0.4 | 0.00 ± 0.01 | na |
| (SEQ ID NO: 2) | | | | |
| M43C | 354 ± 3 | 32 ± 2 | −0.17 ± 0.03 | 1.13 |
| (SEQ ID NO: 11) | | | | |
| N45C | 364 ± 1 | 27 ± 4 | −0.27 ± 0.09 | 1.03 |
| (SEQ ID NO: 12) | | | | |
| F46C | 8 ± 1 | >1000 | >2 | 1.04 |
| (SEQ ID NO: 13) | | | | |
| V48C | 264 ± 3 | 73 ± 7 | 0.31 ± 0.06 | 1.22 |
| (SEQ ID NO: 14) | | | | |
| Y56C | 4.2 ± 0.1 | >1000 | >2 | 1.10 |
| (SEQ ID NO: 15) | | | | |
| W57C | 5.3 ± 0.2 | >1000 | >2 | 1.02 |
| (SEQ ID NO: 16) | | | | |
| S76C | 468 ± 4 | 19 ± 4 | −0.5 ± 0.1 | 1.02 |
| (SEQ ID NO: 17) | | | | |
| A78C | 313 ± 2 | 31 ± 2 | −0.19 ± 0.03 | 0.58 |
| (SEQ ID NO: 18) | | | | |
| T79C | 56 ± 1 | 257 ± 9 | 1.06 ± 0.02 | 0.81 |
| (SEQ ID NO: 19) | | | | |
| D81C | 53 ± 1 | 290 ± 59 | 1.1 ± 0.1 | 0.94 |
| (SEQ ID NO: 20) | | | | |
| K89C | 239 ± 1 | 60 ± 4 | 0.20 ± 0.04 | 1.05 |
| (SEQ ID NO: 21) | | | | |
| W90C | 25 ± 1 | 972 ± 185 | 1.8 ± 0.1 | 0.73 |
| (SEQ ID NO: 22) | | | | |

Properties of cysteine mutants of DarpMbp3_{—}16. The experiments were performed at 25° C. in buffer M3. WT, wild type DarpMbp3_{—}16; R_{eq}, Biacore signal at equilibrium for the binding of the Darpin to immobilized bt-MalE; K_{d}, dissociation constant between the Darpin and MalE, as measured in solution by competition Biacore; ΔΔG, variation of the free energy of interaction between DarpMbp3_{—}16 and MalE, resulting from the mutation; y_{c}, coupling yield of IANBD to the mutant Darpin, i.e. number of molecules of fluorophore per molecule of DarpMbp3_{—}16 in a purified preparation of the conjugate. The mean value and standard error SE are given for R_{eq} in two independent experiments; for the K_{d} of the wild type Darpin in four independent experiments; for the K_{d}s of the mutant Darpins in the fitting of the equilibrium equation to the experimental data; and for ΔΔG as deduced from SE on the K_{d} values (equations 17 and 18).

As explained previously each of these twelve mutants correspond to the fully variable residues of the designed ankyrin repeat as follows in Table 6 below:

TABLE 6

| Residue in DarpMbp3_16 | Residue in ankyrin repeat |
|---|---|
| M43C (SEQ ID NO: 11) | 43^{rd} of N-terminal capping ankyrin repeat |
| | (the 43^{rd} residue of the N-terminal |
| | capping ankyrin repeat is equivalent to |
| | the 33^{rd} residue of the desgined ankyrin |
| | repeat) |
| N45C (SEQ ID NO: 12) | 2^{nd} of designed 1^{st} ankyrin repeat |
| F46C (SEQ ID NO: 13) | 3^{rd} of designed 1^{st} ankyrin repeat |
| V48C (SEQ ID NO: 14) | 5^{th} of designed 1^{st} ankyrin repeat |
| Y56C (SEQ ID NO: 15) | 13^{th} of designed 1^{st} ankyrin repeat |
| W57C (SEQ ID NO: 16) | 14^{th} of designed 1^{st} ankyrin repeat |
| S76C (SEQ ID NO: 17) | 33^{rd} of designed 1^{st} ankyrin repeat |
| A78C (SEQ ID NO: 18) | 2^{nd} of designed 2^{nd} ankyrin repeat |
| T79C (SEQ ID NO: 19) | 3^{rd} of designed 2^{nd} ankyrin repeat |
| D81C (SEQ ID NO: 20) | 5^{th} of designed 2^{nd} ankyrin repeat |
| K89C (SEQ ID NO: 21) | 13^{th} of designed 2^{nd} ankyrin repeat |
| W90C (SEQ ID NO: 22) | 14^{th} of designed 2^{nd} ankyrin repeat |

Shows the correspondence of the residues varied by the inventors in DarpMbp3_{—}16 to the variable residues of the designed ankyrin repeats which it comprises.

The inventors chemically treated the purified preparations of the twelve DarpMbp3_{—}16 cysteine mutants to a reduction reaction before coupling with IANBD, to break open any intermolecular disulfide bonds and ensure that the cysteine would be in a reactive state to receive the flurophore.

The products of the coupling reaction were separated from unreacted fluorophore by chromatography on a nickel ion column. The yield of coupling was calculated from the absorbance spectra of the purified reaction product (see example 1. Materials and Methods) and was found to be close to 100% for nine of the twelve DarpMbp3_{—}16 mutants and lower for the mutants at positions Ala78 (58%), Thr79 (81%) and Trp90 (73%) (Table 5).

These variations in coupling have already been observed for other proteins and in particular DarpOff7 (Example 2). The synthesis yield of the coupling procedure, i.e. the proportion of protein molecules that survived the procedure, was close for all the DarpMbp3_{—}16 mutants, 70.8±2.0% (mean±SE).

### 3.3 Cysteine Scanning of the Randomized Positions

The inventors characterized the properties of recognition between the Cys mutants of DarpMbp3_{—}16 and MalE by two methods, using the Biacore instrument. This characterization was performed in the presence of DTT (5 mM) to eliminate any adduct with the mutant cysteine and intermolecular disulfide bond.

In a preliminary experiment, the inventors immobilized bt-MaIE, a biotinylated form of MalE, on a streptavidine chip, then introduced each of DarpMbp3_{—}16(wt) and its mutant derivatives onto the chip at a fixed concentration (50 nM) in the liquid phase, and measured the variation of resonance signal at equilibrium R_{eq} with a Biacore instrument. The R_{eq} value for DarpMbp3_{—}16 (wt) was equal to 400±2 RU (resonance units). The four mutations that changed aromatic residues, F46C, Y56C, W57C and W90C, strongly decreased the value of R_{eq}, below 25 RU. The other mutations affected R_{eq} to varying extent, with values comprised between 53 and 468 RU.

Except for the mutants at positions 46, 56 and 57, the R_{eq} values were large enough to allow the determination of the dissociation constant K_{d} between the mutant Darpins and MalE by competition Biacore in solution (FIG. 11; Table 5).

In FIG. 11 the results of experiments conducted to determine the dissociation constant between DarpMbp3_{—}16(wt) and MalE by competition Biacore in solution are shown. The total concentration of MalE in the binding reaction is given along the x axis. The r signal, which is proportional to the concentration of free DarpMbp3_{—}16 in the binding reaction, is given along the y axis. Fifteen concentrations of MalE were used. The curve was obtained by fitting the equation of the equilibrium to the experimental data, with K_{d} and r₀ as floating parameters (see Example 1. Materials and Methods).

The mutant Darpin, DarpMbp3_{—}16 (W90C), had a R_{eq} value equal to 25±1 RU and a K_{d} value equal to 972±185 nM. These values indicated that the mutant Darpins with R_{eq}<25 RU had K_{d} values larger than 1000 nM (see below). Therefore, four mutations, changing aromatic residues and listed above, decreased the free energy of association between DarpMbp3_{—}16 and MalE strongly, by more than 1.5 kcal mol⁻¹. Two other mutations, changing residues Thr79 and Asp81, decreased this free energy significantly, by 1.1 kcal mol⁻¹. The six other mutations either did not change the free energy of association or increased it slightly.

Thus, the paratope of DarpMbp3_{—}16 is _mainly formed by a tight cluster of six residues, at the randomized positions 46, 56, 57, 79, 81 and 90. These six residues therefore correspond to the R₁ residues which make up the paratope.

### 3.4 Mapping a Darpin Paratope by Cysteine Scanning

As the residues of a Darpin that contribute to the recognition of its target, are located mainly at the randomized positions by design, to identify the paratope (target binding site) of DarpMbp3_{—}16, the inventors therefore following their rational design strategy changed the residues of its randomized positions individually into cysteine, and measured the K_{d} values between the corresponding mutant proteins and their MalE target.

Six among the 12 mutations decreased the free energy of interaction between DarpMbp3_{—}16 and MalE. Four of the corresponding residues, Phe46, Tyr56, Trp57 and Trp90 were aromatic and constituted hotspots of binding energy (ΔΔG≧1.5 kcal mol⁻¹). Two polar residues, Thr79 and Asp81 contributed significantly to the interaction (ΔΔG≧1.0 kcal mol⁻¹). These six residues formed a tight cluster of residues at the surface of the canonical Darpin structure.

They constituted the set R1 of randomized positions that have an energetic importance for the interaction between DarpMbp3_{—}16 and its target, and at least part of its energetic paratope.

The experimental values of R_{eq} and K_{d} were related by the theoretical equation 1 with a high correlation factor R=0.97 (FIG. 12).

In FIG. 12, the results of experiments to determine the relationship between R_{eq} and K_{d} for the interaction between the DarpMbp3_{—}16 mutants and MalE are shown. The values of R_{eq} and K_{d} were determined by Biacore (see Table 5). The curve was obtained by fitting equation 1 to the experimental values of R_{eq} and K_{d}, with Rₘₐₓ as a floating parameter and C=[DarpMbp3_{—}16]=50 nM. The values of the Pearson coefficient R in the fitting and Rₘₐₓ were equal to 0.96524 and 595±27 RU respectively

Therefore, the measurement of the R_{eq} values for cysteine mutants of a Darpin enables one to rapidly and reliably characterize the subset of randomized positions that are important or not for the interaction with its target.

### 3.5 Fluorescence Properties of the Conjugates

The free conjugates were excited at 485 nm and their emission spectra were recorded. The maximums of fluorescence intensity had wavelengths λₘₐₓ that varied slightly between conjugates, from 535 to 540 nm (Table 7). The following experiments of fluorescence were performed at the λₘₐₓ value for each conjugate.

In the present Patent Application so as to fully validate the new rational design method proposed, R₁ residues were studied further so as to confirm that these residues were not suitable to conjugate a fluorophore too. Such further work would not normally be necessary following the proposed rational design method.

The inventors tested the responsiveness of the DarpMbp3_{—}16 conjugates to the binding of their MalE target by measuring the relative variation ΔF/F₀=(F−F₀)/F₀ in their fluorescence intensity F between their MalE-bound and free states.

The concentration of conjugate was chosen to fulfil the following requirements. (i) The fluorescence intensity F₀ of the free conjugate had to be higher than the background signal of the measurement and within the dynamic interval of the spectrofluorometer. (ii) The dynamic interval of the measurements had to cover more than one order of magnitude in target concentration for a conjugate that would have the same dissociation constant K_{d} as the parental Darpin.

The inventors chose a concentration of conjugate equal to 1 μM in these experiments, since K_{d} between DarpMbp3_{—}16 and MalE was equal to 43 nM (see below). The values of F₀ for the various conjugates were comprised between 6.8 and 42.8 FU (fluorescence units) at this concentration (Table 7).

TABLE 7

| | λₘₐₓ | F₀ | | ~K_{d} | |
|---|---|---|---|---|---|
| Residue | (nm) | (FU) | ~ΔF_{∞}/F₀ | (μM) | ~sᵣ(1 μM) |
| Met43 | 540 | 42.8 ± 0.6 | 0.49 | 0.19 | 0.4 |
| Asn45 | 539 | 24.2 ± 0.1 | 1.7 | 0.20 | 1.4 |
| Phe46 | 538 | 12.8 ± 0.1 | 1.6 | 6.5 | 0.2 |
| Val48 | 538 | 19.6 ± 0.4 | 0.12 | 0.56 | 0.1 |
| Tyr56 | 535 | 17.7 ± 0.1 | 0.24 | 8.0 | 0.0 |
| Trp57 | 535 | 15.5 ± 0.1 | 3.8 | 22 | 0.2 |
| Ser76 | 538 | 33.5 ± 0.1 | 0.67 | 0.085 | 0.6 |
| Ala78 | 538 | 15.7 ± 0.2 | 1.0 | 0.13 | 0.9 |
| Thr79 | 537 | 9.2 ± 0.1 | 5.6 | 18 | 0.3 |
| Asp81 | 536 | 7.3 ± 0.1 | 6.8 | 20 | 0.3 |
| Lys89 | 535 | 6.8 ± 0.1 | 6.0 | 0.63 | 3.7 |
| Trp90 | 538 | 9.6 ± 0.1 | 1.4 | 10 | 0.1 |

Ranking of the DarpMbp3_{—}16 conjugates by comparison of their fluorescence properties in screening experiments. The experiments were performed at 25° C. in buffer M1 with a concentration of conjugate that was fixed and equal to 1 μM. Column 1 gives the residue of DarpMbp3_{—}16 that was mutated into Cys and then coupled with IANBD. F₀, maximal intensity of light emission by the free conjugate on excitation at 485 nm; λₘₐₓ, wavelength at which F₀ was attained; ˜ΔF_{∞}/F₀, maximal variation of fluorescence intensity at λₘₐₓ for the conjugate on target binding, as determined by fitting equation 8 to a minimal experiment of titration which included only three concentrations of MalE (0 μM, 1 μM and 10 μM); ˜K_{d}, dissociation constant between the conjugate and MalE in the same minimal experiment; ˜sᵣ(1 μM), relative sensitivity of the conjugate at its fixed concentration of 1 μM, as deduced from the values of ˜ΔF_{∞}/F₀ and ˜K_{d} by equation 11. The mean value of F₀ and associated SE in two independent experiments are given.

The inventors measured ΔF/F₀ for all the conjugates at three concentrations of MalE, 0 μM, 1 μM and 10 μM, as a first screen. The theoretical equation 8, linking ΔF/F₀ and the concentration of target, was fitted to these minimal experimental data with ΔF_{∞}/F₀ and K_{d} as floating parameters (Example 1. Materials and Methods). The approximate values ˜ΔF_{∞}/F₀ and ˜K_{d} thus obtained are given in Table 7. ˜ΔF_{∞}/F₀ varied between 0.12 and 6.8 and ˜K_{d} between 0.086 and 22 μM according to the conjugate. The inventors calculated an approximate value ˜sᵣ(1 μM) of the relative sensitivity of the conjugates at a concentration of 1 μM from ˜ΔF_{∞}/F₀ and ˜K_{d} to rank them (equation 11). The values of ˜sᵣ(1 μM) varied between 0.1 and 3.7 according to the conjugate (Table 7). A value of sᵣ lower than one, means that the relative variation of fluorescence ΔF/F₀ increases less rapidly that the degree of occupation of the conjugate by its target for the low concentrations of target.

The inventors chose to study in more details the conjugates whose ˜sᵣ(1 μM) value was higher than or equal to 0.4, i.e. those at positions Met43, Asn45, Ser76, Ala78 and Lys89. The titration of the conjugates by the target was repeated with ≧14 concentrations of MalE (FIG. 10).

In FIG. 10 the titration of DarpMbp3_{—}16 conjugates by MalE was monitored by fluorescence. The experiments were performed at 25° C. in buffer M1. The total concentration in DarpMbp3_{—}16, as measured by A_{280 nm}, was equal to 1 μM. The total concentration in MalE protein is given along the x axis; a data point at 10 μM is not shown on the figure. The continuous curves correspond to the fitting of equation 8 to the experimental values of ΔF/F₀ (Example 1. Materials and Methods). (Δ) position Met43; (◯) Asn45; (▴) Ala78; () Lys89.

The corresponding accurate values of ΔF_{∞}/F₀ and K_{d} are given in Table 8.

TABLE 8

| | f_{b} | | K_{d} | ΔΔG |
|---|---|---|---|---|
| Residue/Group | (FU μM⁻¹) | ΔF_{∞}/F₀ | (μM) | (kcal mol⁻¹) |
| Met43/R₃ | 37.9 ± 0.5 | 0.46 ± 0.01 | 0.15 ± 0.04 | 0.8 ± 0.1 |
| Asn45/R₂ | 23.5 ± 0.1 | 1.69 ± 0.03 | 0.25 ± 0.03 | 1.0 ± 0.1 |
| Ser76/R₂ | 32.8 ± 0.1 | 0.65 ± 0.01 | 0.08 ± 0.02 | 0.4 ± 0.1 |
| Ala78/R₂ | 27.0 ± 0.3 | 1.00 ± 0.03 | 0.20 ± 0.04 | 0.9 ± 0.1 |
| Lys89/R₂ | 6.5 ± 0.1 | 5.8 ± 0.1 | 0.69 ± 0.06 | 1.6 ± 0.1 |

Detailed properties of selected DarpMbp3_{—}16 conjugates, as derived from fluorescence experiments. The experiments were performed in the conditions of FIG. 10. Column 2 gives the molar fluorescence f_{b} of the free conjugates, as deduced from the values of F₀ and y_{c} (Tables 5 and 7). The entries for ΔF_{∞}/F₀ and K_{d} give the values of these two parameters and associated SE in the fitting of equation 8 to the data points. ΔΔG, variation of the free energy of interaction between DarpMbp3_{—}16 and MalE, resulting from the presence of the fluorescent group. The value of K_{d} for DarpMbp3_{—}16(wt) was obtained by experiments of competition Biacore in solution and is given in Table 5. The values of SE on ΔΔG were calculated from the values of SE on K_{d} (Example 1. Materials and Methods). The groups to which the selected conjugates belong R₁ or R₃ is also shown.

Remarkably, the accurate values, obtained from detailed experiments, and the approximate values, obtained from minimal experiments, were very close. The difference between ˜K_{d} and K_{d} was lower than 1.5-fold, and that between ˜ΔF_{∞}/F₀ and ΔF_{∞}/F₀ was lower than 1.1-fold. Therefore, the minimal experiment appeared as a reliable and rapid method to screen the fluorescent conjugates with the best properties.

### 3.6 Ranking of the Conjugates

The inventors ranked the conjugates of DarpMbp3_{—}16 according to their relative sensitivity s₁ and their absolute sensitivity s (equations 11 and 12). The ranking according to sᵣ, of the five conjugates that the inventors studied in detail, was the following when their concentration was higher than 0.16 μM: Met43<Ser76<Ala78<Asn45<Lys89 (Tables 7 and 8). The fluorescence signal F increased 3.5 fold faster that the occupation of the conjugate by its target, both in relative terms, for low concentrations of MalE and for the DarpMbp3_{—}16(K89ANBD) conjugate at a concentration of 1.0 μM.

The inverse s⁻¹ of the absolute sensitivity relates the lower limit of detection for a conjugate to the lower limit of measurement for the spectrofluorometer, which are proportional for the low concentrations of target. The ranking of the five above conjugates, according to s⁻¹ and therefore to their lower limit of detection, was the following when their concentration was higher than 1 μM: Asn45<Lys89<Ala78<Ser76<Met43. The lower limit of detection for the DarpMbp3_{—}16(N45ANBD) conjugate was equal to 32 nM FU⁻¹. The lower limits of detection and the corresponding ranking of the conjugates varied widely as a function of their concentration below 1.0 μM (FIG. 13).

FIG. 13 shows the ranking of DarpMbp3_{—}16 conjugates according to their lower limit of detection at 25° C. in buffer M1. The s⁻¹ parameter gives the lower concentration of target [A]₀ that can be detected by a conjugate, when the lower variation of fluorescence intensity that can be detected by the spectrofluorometer, is equal to 1 FU. (Δ) position Met43; (◯) Asn45; (♦) Ser76; (▴) Ala78; () Lys89.

### 3.7 Validity of the Design Rule

FIG. 14 summarizes and compares the experimental data that the inventors obtained for each of the fully randomized positions of DarpMbp3_{—}16.

FIG. 14 shows the relative positions of the coupling sites in the ankyrin repeats. AR1 and AR2, ankyrin repeats 1 and 2 respectively. Positions 2, 3, 5, 13, 14, and 33 in each ankyrin repeat are fully randomized and represented in roman type. Position 26 in each repeat is partially randomized and represented in underlined type. The positions in the N-cap and C-cap that are structurally equivalent to the above positions but are not randomized, are represented in italic type. Position 43 in the N-cap module is fully randomized and position 109 in AR2 is not randomized (Binz et al. 2003). The figure gives the corresponding residues in the sequence of DarpMbp3_{—}16. ΔΔG₁, variation of the free energy of interaction between DarpMbp3_{—}16 and MalE resulting from the mutation into Cys; ΔΔG₂, variation of energy resulting from the coupling of the fluorophore; sᵣ(1), relative sensitivity of the conjugate at a concentration of 1 μM. Data from Tables 5, 7 and 8.

The coupling of the IANBD fluorophore at a randomized position was detrimental to the interaction between the Darpin and its target (ΔΔG2>0) to various degrees. The coupling of a fluorophore increased the deleterious effects of the mutations into cysteine (ΔΔG2>ΔΔG1), with the possible exception of the aromatic residue Tyr56.

The most sensitive conjugates (sᵣ(1 μM)>0.8) corresponded to three positions, Asn45, Ala78 and Lys89, of DarpMbp3_{—}16 that were not important for the interaction with the antigen (ΔΔG1≦0.2 kcal mol-1) but in position −1 along the sequence relative to important positions of the same DarpMbp3_{—}16. Several conjugates corresponded to residues of DarpMbp3_{—}16 that were not important for the interaction with the antigen but predicted in contact with important residues of the same DarpMbp3_{—}16 from the structure of the canonical Darp3_{—}5: e. g. Ala78 would be in contact with Asp46, and Ser76 with Asp81 (FIG. 9).

FIG. 9 shows the randomized positions in the crystal structure of DarpE3_{—}5. The ankyrin repeats are represented in alternating light grey and dark grey, with the N-cap on top. The randomized positions are numbered, and equivalent positions in the sequence of the Darpin have the same colour, light or dark according to the repeat. The visible positions are fully randomized (all residues except Gly, Cys or Pro). Positions 69, 102, 135, with partial randomization (Asn, His or Tyr), are not visible. Analysis of the structure showed that the following couples of residues are in direct contact: Thr43-Tyr48, Asn45-Leu78, Asp46-Tyr48, Asp46-Leu78, Ser56-Thr90, Ser76-Ile81, Leu78-Asn111, Thr79-Ile81, Thr79-Asn111, Ala89-Tyr123, Tyr109-His114 and Asp112-His114.

Based upon this structure the inventors were able to validate that the residues Ala78 and Ser76 are in contact with residues of the paratope Asp46 and Asp81 respectively.

Finally, a sensitive conjugate corresponded to residue Met43 of DarpMbp3_{—}16, which occupied a randomized position but was neither important for the interaction with the antigen nor predicted adjacent to important residues of the same DarpMbp3_{—}16.

### 3.8 Conclusions

The design rules that the inventors have developed, have been validated by the experiments with the model Darpin, DarpMbp3_{—}16.

These rules consist of:

(i) focusing the search of target positions for the coupling of fluorophores to the randomized positions (set R);

(ii) avoiding the positions of subset R₁ that contribute to the free energy of interaction with the target;

(iii) favoring the positions of subset R₂ that do not contribute to the interaction and are adjacent to residues that do contribute, either along the sequence (i.e. in positions n−1 or n+1; e. g. Asn45, Ala78, Lys89) or in the canonical structure of Darpins (i.e. in contact via Van der Waals bonds; e. g. Ser76, Ala78);

(iv) using the other positions that do not contribute to the interaction and constitute subset R₃ (e. g. Met43), if no solution is found within subset R₂

(v) coupling a flurophore to at least one R₂ or R₃ residue.

The inventors showed that it is possible to characterize the randomized positions that are important for the interaction with the target, rapidly by mutations into cysteine and measurement of the R_{eq} signal in experiments of binding monitored by Biacore. The inventors also showed that it is possible to characterize and compare the properties of the conjugates (ΔF_{∞}/F₀, K_{d} and sᵣ) by minimal experiment of titration of the conjugate by the target.

The inventors results showed that it is possible to obtain reagentless fluorescent biosensors from any Darpin and in the absence of the structure between the Darpin and its target. The inventors also described simple and fast methods to obtain them.

### REFERENCES

Altschuh, D., Oncul, S., and Demchenko, A. P. 2006. Fluorescence sensing of intermolecular interactions and development of direct molecular biosensors. J Mol Recognit 19: 459-477.

Amstutz, P., Binz, H. K., Parizek, P., Stumpp, M. T., Kohl, A., Grutter, M. G., Forrer, P., and Pluckthun, A. 2005. Intracellular kinase inhibitors selected from combinatorial libraries of designed ankyrin repeat proteins. J Biol Chem 280: 24715-24722.

Binz, H. K., Amstutz, P., Kohl, A., Stumpp, M. T., Briand, C., Forrer, P., Grutter, M. G., and Pluckthun, A. 2004. High-affinity binders selected from designed ankyrin repeat protein libraries. Nat Biotechnol 22: 575-582.

Binz, H. K., Amstutz, P., and Pluckthun, A. 2005. Engineering novel binding proteins from nonimmunoglobulin domains. Nat Biotechnol 23: 1257-1268.

Binz, H. K., Stumpp, M. T., Forrer, P., Amstutz, P., and Pluckthun, A. 2003. Designing repeat proteins: well-expressed, soluble and stable proteins from combinatorial libraries of consensus ankyrin repeat proteins. J Mol Biol 332: 489-503.

Bullock, W. O., Fernandez, J. M., and Short, J. M. 1987. XL1-Blue: a high efficiency plasmid transforming recA Escherichia coli strain with beta-galactosidase selection. BioTechniques 5: 376-379.

de Lorimier, R. M., Smith, J. J., Dwyer, M. A., Looger, L. L., Sali, K. M., Paavola, C. D., Rizk, S. S., Sadigov, S., Conrad, D. W., Loew, L., et al. 2002. Construction of a fluorescent biosensor family. Protein Sci 11: 2655-2675.

de Lorimier, R. M., Tian, Y., and Hellinga, H. W. 2006. Binding and signaling of surface-immobilized reagentless fluorescent biosensors derived from periplasmic binding proteins. Protein Sci 15: 1936-1944.

England, P., Bregegere, F., and Bedouelle, H. 1997. Energetic and kinetic contributions of contact residues of antibody D1.3 in the interaction with lysozyme. Biochemistry 36: 164-172.

Foote, J., and Winter, G. 1992. Antibody framework residues affecting the conformation of the hypervariable loops. J Mol Biol 224: 487-499.

Gelin B. R. et al., Biochemistry, 1979, 18, 1256-1268.

Griffiths, D., and Hall, G. 1993. Biosensors—what real progress is being made? Trends Biotechnol 11: 122-130.

Harlow, E., and Lane, D. 1988. Antibodies: a laboratory manual. Cold Spring Harbor Laboratory, Cold Spring Harbor, N.Y., pp. xiii, 726 p.

Huber, T., Steiner, D., Rothlisberger, D., and Pluckthun, A. 2007. In vitro selection and characterization of DARPins and Fab fragments for the co-crystallization of membrane proteins: The Na(+)-citrate symporter CitS as an example. J Struct Biol 159: 206-221.

Kawe, M., Forrer, P., Amstutz, P., and Pluckthun, A. 2006. Isolation of intracellular proteinase inhibitors derived from designed ankyrin repeat proteins by genetic screening. J. Biol. Chem. 281: 40252-40263. P5

Jespers, L., Bonnert, T. P., and Winter, G. 2004. Selection of optical biosensors from chemisynthetic antibody libraries. Protein Eng Des Sel 17: 709-713.

Kohl, A., Binz, H. K., Forrer, P., Stumpp, M. T., Pluckthun, A., and Gruffer, M. G. 2003. Designed to be stable: crystal structure of a consensus ankyrin repeat protein. Proc Natl Acad Sci USA 100: 1700-1705.

Lakowicz, J. R. 1999. Principles of fluorescent spectroscopy, 2nd ed. Kluwer Academic/Plenum, New York.

Li, J., Mahajan, A., and Tsai, M. D. 2006. Ankyrin repeat: a unique motif mediating protein-protein interactions. Biochemistry 45: 15168-15178.

Lisova, O., Hardy, F., Petit, V., and Bedouelle, H. 2007. Mapping to completeness and transplantation of a group-specific, discontinuous, neutralizing epitope in the envelope protein of dengue virus. J Gen Virol 88: 2387-2397.

Lowe, C. R. 1984. Biosensors. Trends Biotechnol 2: 59-65.

Mathonet, P., and Fastrez, J. 2004. Engineering of non-natural receptors. Curr Opin Struct Biol 14: 505-511.

Morgan, C. L., Newman, D. J., and Price, C. P. 1996. Immunosensors: technology and opportunities in laboratory medicine. Clin Chem 42: 193-209.

Mosavi, L. K., Cammett, T. J., Desrosiers, D. C., and Peng, Z. Y. 2004. The ankyrin repeat as molecular architecture for protein recognition. Protein Sci 13: 1435-1448.

Mosavi, L. K., Minor, D. L., Jr., and Peng, Z. Y. 2002. Consensus-derived structural determinants of the ankyrin repeat motif. Proc Natl Acad Sci USA 99: 16029-16034.

Nieba, L., Krebber, A., and Pluckthun, A. 1996. Competition BIAcore for measuring true affinities: large differences from values determined from binding kinetics. Anal Biochem 234: 155-165.

Pace, C. N., Vajdos, F., Fee, L., Grimsley, G., and Gray, T. 1995. How to measure and predict the molar absorption coefficient of a protein. Protein Sci 4: 2411-2423.

Renard, M., and Bedouelle, H. 2004. Improving the sensitivity and dynamic range of reagentless fluorescent immunosensors by knowledge-based design. Biochemistry 43: 15453-15462.

Renard, M., Belkadi, L., and Bedouelle, H. 2003. Deriving topological constraints from functional data for the design of reagentless fluorescent immunosensors. J Mol Biol 326: 167-175.

Renard, M., Belkadi, L., Hugo, N., England, P., Altschuh, D., and Bedouelle, H. 2002. Knowledge-based design of reagentless fluorescent biosensors from recombinant antibodies. J Mol Biol 318: 429-442.

Rich, R. L., and Myszka, D. G. 2005. Survey of the year 2004 commercial optical biosensor literature. J Mol Recognit 18: 431-478.

Rondard, P., and Bedouelle, H. 1998. A mutational approach shows similar mechanisms of recognition for the isolated and integrated versions of a protein epitope. J Biol Chem 273: 34753-34759.

Sheriff S. et al., J. Mol. Biol., 1987, 197, 273-296.

Sheriff S., ImmunoMethods, 1993, 3, 191-196.

Smith, J. J., Conrad, D. W., Cuneo, M. J., and Hellinga, H. W. 2005. Orthogonal site-specific protein modification by engineering reversible thiol protection mechanisms. Protein Sci 14: 64-73.

Smith, P. A., Tripp, B. C., DiBlasio-Smith, E. A., Lu, Z., LaVallie, E. R., and McCoy, J. M. 1998. A plasmid expression system for quantitative in vivo biotinylation of thioredoxin fusion proteins in Escherichia coli. Nucleic Acids Res 26: 1414-1420.

Sowdhamini, R., Srinivasan, N., Shoichet, B., Santi, D. V., Ramakrishnan, C., and Balaram, P. 1989. Stereochemical modeling of disulfide bridges. Criteria for introduction into proteins by site-directed mutagenesis. Protein Eng 3: 95-103.

Tatutsova and Madden, FEMS Microbiol Lett., 1999, 174, 247-250

Thevenot, D. R., Toth, K., Durst, R. A., and Wilson, G. S. 2001. Electrochemical biosensors: recommended definitions and classification. Biosens Bioelectron 16: 121-131.

Vessman, J., Stefan, R. I., Van Staden, J. F., Danzer, K., Lindner, W., Burns, D. T., Fajgelj, A., and Muller, H. 2001. Selectivity in analytical chemistry—(IUPAC Recommendations 2001). Pure and Applied Chemistry 73: 1381-1386.

Vo-Dinh, T., and Kasili, P. 2005. Fiber-optic nanosensors for single-cell monitoring. Anal Bioanal Chem 382: 918-925.

Vriend, G. 1990. WHAT IF: a molecular modeling and drug design program. J Mol Graph 8: 52-56, 29.

Zahnd, C., Amstutz, P., and Pluckthun, A. 2007. Ribosome display: selecting and evolving proteins in vitro that specifically bind to a target. Nat Methods 4: 269-279.

Zahnd, C., Pecorari, F., Straumann, N., Wyler, E., and Pluckthun, A. 2006. Selection and characterization of Her2 binding-designed ankyrin repeat proteins. J Biol Chem 281: 35167-35175.

Zhu, H., and Snyder, M. 2003. Protein chip technology. Curr Opin Chem Biol 7: 55-63.

1. A reagentless peptide biosensor for at least one ligand, comprising:
at least one ankyrin repeat module;
at least one cysteine residue coupled to a fluorophore.

2. The biosensor of claim 1, wherein the cysteine residue is present at a position of said biosensor whose solvent accessible surface area is altered when said biosensor binds to said at least one ligand but which does not directly interact therewith.

3. The biosensor of claim 1, wherein at least one ankyrin repeat of said at least one ankyrin repeat module consists of SEQ ID NO: 30 or SEQ ID NO: 7, or a sequence of at least 60% similarity therewith.

4. The biosensor of claim 3, wherein said fluorophore is coupled to one residue of SEQ ID NO: 30 or SEQ ID NO: 7 selected from:
(i) residues 2, 3, 5, 13, 14, 26 and 33; or
(ii) residues 1, 4, 6, 12, 15, 25, 27, 32.

5. The biosensor of claims 1, comprising at least an N-terminal capping ankyrin repeat and/or a C-terminal capping ankyrin repeat.

6. The biosensor of claim 5, wherein said N-terminal capping ankyrin repeat consists of SEQ ID NO: 8 or SEQ ID NO: 23 and said C-terminal capping ankyrin repeat consists of SEQ ID NO: 10 or SEQ ID NO: 24.

7. The biosensor of claim 1, wherein said at least one cysteine residue is either present in said biosensor or is substituted for another suitable residue whose solvent accessible surface area alters when said biosensor binds to said ligand but which does not directly interact therewith.

8. The biosensor of claims 1, wherein said at least one residue forms an indirect contact with said ligand via at least one water molecule.

9. The biosensor of claim 1, wherein said at least one residue does not contact said ligand.

10. The biosensor of claim 1, comprising more than one ankyrin repeat module.

11. The biosensor of claim 10, wherein a residue in a second domain corresponding to a contacting residue in a first domain, is coupled to a fluorophore.

12. The biosensor as claimed in claim 1, wherein said fluorophore is selected from the group consisting of: IANBD, CNBD, acrylodan, 5-iodoacetamidofluorescein or a fluorophore having an aliphatic chain of 1 to 6 carbon atoms.

13. The biosensor as claimed in claim 1, wherein said biosensor is in soluble form.

14. The biosensor as claimed in claim 1, wherein said biosensor is immobilized on a suitable solid support.

15. The biosensor as claimed in claim 1, wherein said biosensor consists of SEQ ID NO: 28 in which at least one of residues 23, 45, 46, 53, 111, 112, 114, 122, 123 and 125 have been substituted with a cysteine residue and coupled to said fluorophore.

16. A protein-based chip, characterized in that it consists of a solid support on which at least one biosensor as claimed in claim 1 is immobilized.

17. A solution comprising at least one biosensor as claimed in claim 1.

18. An optical fibre comprising at a first end thereof at least one biosensor as claimed in claim 1 and comprising at a second end thereof means to attach said optical fibre to a device configured to receive an interpret the output of said at least one biosensor.

19. A method for producing biosensors as claimed in claim 1, characterized in that it comprises the following steps:
(a) selecting at least one residue of the biosensor by searching for the residues which have a solvent accessible surface area (ASA) which is modified by the binding of said at least one ligand, when use is made of spheres of increasing radius of 1.4 to 30 Å, for the molecule of said solvent; and which (i) are in contact with said ligand via a water molecule, or (ii) do not contact said ligand;
(b) mutating by site-directed mutagenesis at least one of the residues selected in (a) to a Cys residue when said residue is not naturally a Cys residue, and
(c) coupling the Sγ atom of at least one Cys residue obtained in (a) or in (b) to a fluorophore.

20. The preparation method as claimed in claim 19, characterized in that, prior to step (a), it comprises a step of modelling the biosensor and/or the ligand and/or the biosensor-ligand complex.

21-24. (canceled)

25. A reagent for detecting, assaying or locating ligands, characterized in that it includes at least one biosensor as claimed in claim 1.

26. A method for detecting, assaying or locating a ligand in a heterogeneous sample, characterized in that it comprises bringing said heterogeneous sample into contact with at least one reagent as claimed in claim 25.

27. A kit for detecting, assaying or locating ligands, characterized in that it includes at least one reagent as claimed in claim 25.

28. A kit for screening for inhibitors of the ligand/receptor interaction, characterized in that it includes at least one reagent as claimed in claim 25.

29. A reagentless peptide biosensor for at least one ligand, wherein said biosensor comprises at least two ankyrin repeat modules and each of said ankyrin repeat modules comprises at least two cysteine residues, and wherein a fluorophore is attached to a first cysteine residue in each of said ankyrin repeat modules, and wherein each of said ankyrin repeat modules is linked to at least one other of said ankyrin repeat modules via a disulfide bond between a second cysteine residue in each of said ankyrin repeat modules.

30. The reagentless biosensor of claim 29, wherein said at least two ankyrin repeat modules are homologous.

31. The reagentless biosensor of claim 29, wherein said at least two ankyrin repeat modules are heterologous.

32. The reagentless biosensor of claim 31, wherein each of said heterologous ankyrin repeat modules comprise a different fluorophore.

33. The biosensor of claim 29, wherein each said first cysteine residue is present at a position of each said ankyrin repeat module whose solvent accessible surface area is altered when said biosensor binds to said at least one ligand but which does not directly interact therewith.

34. A method for preparing reagentless fluorescent biosensors which comprise at least one ankyrin repeat and are specific for at least one target, characterized in that it comprises the following steps:
(a) identifying the residues (R₁) of the paratope of the biosensor by mutagenesis of all, or of a subset, of the residues of the biosensor, and determining variations in at least one measurable chemical or physical parameter of interaction with said at least one target; wherein said variations are due to each mutation or to groups of mutations;
(b) selecting the cysteine residues, or the residues to be mutated to cysteine, from the residues (R₂) of the biosensor which are located adjacent to at least one residue of the paratope (R₁); and/or selecting the cysteine residues, or the residues to be mutated to cysteine, from the residues (R₃) which do not form part of the paratope and which were mutated in step (a);
(c) mutating by site-directed mutagenesis at least one of the residues (R₂) and/or (R₃) selected in (b) to a cysteine residue when said residue is not naturally a cysteine residue; and
(d) coupling the Sγ atom of at least one cysteine residue (R₂) and/or (R₃) obtained in (b) or in (c) to a fluorophore.

35. The method as claimed in claim 34, wherein said at least one measurable chemical or physical parameter is selected from the group: the equilibrium constant (K_{D}) between said biosensor and said at least one target; the dissociation (K_{off}) and/or association (kₒₙ) rate constants for said biosensor and said at least one target; variation of free energy of interaction (ΔΔG) between said biosensor and said at least one target; variation of resonance signal at equilibrium (R_{eq}) between said biosensor and said at least one target.

36. The method of claim 34, wherein in step (b) the selected adjacent residues (R₂) are residues −1 and +1 along the peptide backbone relative to at least one residue of the paratope.

37. The method of claim 34, wherein in step (b) the selected adjacent residues (R₂) are in Van-Der-Waals contact with at least one residue of the paratope.

38. The method as claimed in claim 34, characterized in that, prior to step (a) the nonessential Cys residues of the biosensor are substituted with Ser or Ala residues by site-directed mutagenesis.

39. The method as claimed in claim 34, characterized in that, in step (d), said fluorophore is selected from the group consisting of: IANBD, CNBD, acrylodan, 5-iodoacetamidofluorescein or a fluorophore having an aliphatic chain of 1 to 6 carbon atoms.

40. The method as claimed in claim 34, wherein at least one ankyrin repeat comprises a number of framework residues and a number of variable residues, and said subset of residues of step (a) which are mutated, comprise at least one of said variable residues.

41. The method as claimed in claim 34, wherein at least one ankyrin repeat consists of SEQ ID NO: 7.

42. The method as claimed in claim 41, wherein said subset of residues of step (a) which are mutated are selected from residues 2, 3, 5, 13, 14, 26 and 33 of SEQ ID NO: 7.

43. The method as claimed in claim 34, wherein said biosensor comprises at least an N-terminal capping ankyrin repeat and/or a C-terminal capping ankyrin repeat.

44. The method as claimed in claim 43, wherein said N-terminal capping ankyrin repeat consists of SEQ ID NO: 8 or SEQ ID NO: 23, and said C-terminal capping ankyrin repeat consists of SEQ ID NO: 10 or SEQ ID NO: 24.

45. The method as claimed in claim 44, wherein said subset of residues (R₁) of step (a) also comprises residue 43 of SEQ ID NO: 8 or SEQ ID NO: 23.

46. The method as claimed in claim 34, characterized in that, prior to step (d) the mutated biosensor obtained in step (c) is subjected to a controlled chemical reduction.

47. The method as claimed in claim 34, characterized in that, after step (d), it comprises an additional step (e) of: (e) purifying the biosensor of step (d).

48. The method as claimed in claim 47, characterized in that, after step (e), it comprises an additional step (f) of:
(f) (i) measuring at least one of: the equilibrium constant (K_{D}) between said purified biosensor and said at least one target, or the dissociation (K_{off}) and association (kₒₙ) rate constants for said biosensor and said at least one target; and
(ii) measuring the fluorescence variation of said biosensor between a free and target bound state; and
(g) determining the sensitivity (s) and/or relative sensitivity (sᵣ) of said biosensor from the measurements of step (f) (i) and (ii).

49. The method as claimed in claim 34, characterized in that, after step (d) or step (e) or step (f), it comprises an additional step of immobilizing said biosensor on a solid support.

50. The method as claimed in claim 34, wherein said biosensors comprise at least two ankyrin repeats, characterized in that it comprises the following replacement steps:
(a1) identifying the paratope of a first ankyrin repeat by scanning mutagenesis of the set or of a subset of the residues of said first ankyrin repeat, and determining the variations in the parameters of interaction with the ligand (K_{D}, kₒₙ, k_{off}, ΔΔG, R_{eq}) which are due to each mutation or to limited groups of mutations;
(b1) selecting the Cys residues, or the residues to be mutated into cysteine, from the residues of a second ankyrin repeat which are (i) equivalent to the residues of the paratope, (ii) are located in proximity of the residues of the paratope of said first ankyrin repeat or (iii) are in spatial proximity with the paratope of said first ankyrin repeat;
(c1) mutating by site-directed mutagenesis at least one of the residues selected in (b1) to a Cys residue when said residue is not naturally a Cys residue; and
(d1) coupling the Sγ atom of at least one Cys residue obtained in (b1) or in (c1) to a fluorophore.

51. A biosensor produced according to the method of claims 19.

52. The biosensor of claim 51, comprising a peptide sequence selected from the group: SEQ ID NO: 11, SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22, SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39.

53. A protein-based chip, characterized in that it consists of a solid support on which at least one biosensor as claimed in claim 51 is immobilized.

54. A solution comprising at least one biosensor as claimed in claim 51.

55. An optical fibre comprising at a first end thereof at least one biosensor as claimed in claim 51 and comprising at a second end thereof means to attach said optical fibre to a device configured to receive an interpret the output of said at least one biosensor.


SEQUENCE LISTING (39 sequences)

SEQ ID NO: 1 - DNA, 411 bp, Artificial
  DarpMbp3_16
atgagaggatcgcatcaccatcaccatcacggatccgacctgggtaagaaactgctggaagctgctcatgctggtcaggacgacgaagttcgtatcctgatggctaacggtgctgacgttaacgctatggacaattttggtgttactccgctgcacctggctgcttattggggtcactttgaaatcgttgaagttctgctgaagtacggtgctgacgttaacgcttctgacgctactggtgatactccgctgcaccttgctgctaagtggggttacctgggaatcgttgaagttctgctgaagtacggtgctgacgttaacgctcaggacaaattcggtaagaccgctttcgacatctccatcgacaacggtaacgaggacctggcggaaatcctgcaaaagcttaattag

SEQ ID NO: 2 - protein, 136 aa, Artificial
  DarpM3_16 peptide
MRGSHHHHHHGSDLGKKLLEAAHAGQDDEVRILMANGADVNAMDNFGVTPLHLAAYWGHFEIVEVLLKYGADVNASDATGDTPLHLAAKWGYLGIVEVLLKYGADVNAQDKFGKTAFDISIDNGNEDLAEILQKLN

SEQ ID NO: 3 - DNA, 4570 bp, Artificial
  pQEMBP
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatctggttccatgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggatccggtggtaccccgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 4 - DNA, 5189 bp, Artificial
  pAT224
ctcgagaaatcataaaaaatttatttgctttgtgagcggataacaattataatagattcaattgtgagcggataacaatttcacacagaattcattaaagaggagaaattaactatggctggtctgaacgatatcttcgaagctcagaaaatcgaatggcacgaaggttccatggggaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggatccggtggtaccccgggtcgacctgcagcccaagcttctcatcaccatcaccatcactaatgagctgagcttggactcctgttgatagatccagtaatgacctcagaactccatctggatttgttcagaacgctcggttgccgccgggcgttttttattggtgagaatccaagctagcagtactgcgatgagtggcagggcggggcgtaatttttttaaggcagttattggtgcccttaaacgcctggggtaatgactctctagtttgaggcatcaaataaaacgaaaggctcagtcgaaagactgggcctttcgttttatctgttgtttgtcggtgaacgctctcctgagtaggacaaatccgccgctctagagatttccctcgacaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcacctttcgatggtgtcaacgtaaatgcatgccgcttcgccttccctagctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 5 - DNA, 408 bp, Artificial
  mbp3_161
atgagaggatcgcatcaccatcaccatcacggatccgatctgggtaaaaaactgctggaagctgcacacgctggccaagacgatgaagttcgtatcctgatggcgaacggtgcggatgtaaacgcaatggataactttggcgtgaccccactgcatctggctgcctactggggtcacttcgagattgtggaagtactgctgaaatatggtgcagacgtcaatgcctccgacgcaacgggtgacaccccgctgcacctggccgctaaatggggctacctgggtatcgttgaagttctgctgaagtacggcgcggacgttaacgcgcaggataaattcggtaaaactgctttcgacatctctattgataacggcaacgaagacctggcggagatcctgcagaagcttaat

SEQ ID NO: 6 - protein, 136 aa, Artificial; identical to SEQ ID NO: 2
  DarpMbp3_161 peptide

SEQ ID NO: 7 - protein, 33 aa, Artificial
  Desgined Ankyrin Repeat Consensus
DXXGXTPLHLAAXXGHLEIVEVLLKXGADVNAX

SEQ ID NO: 8 - protein, 43 aa, Artificial
  N-terminal capping ankyrin residue
MRGSHHHHHHGSDLGKKLLEAAHAGQDDEVRILMANGADVNAM

SEQ ID NO: 9 - protein, 65 aa, Artificial
  DarpMbp3_16 desgined ankyrin repeats
DNFGVTPLHLAAYWGHFEIVEVLLKYGADVNASDATGDTPLHLAAKWGYLGIVEVLLKYGADVNA

SEQ ID NO: 10 - protein, 28 aa, Artificial
  C-terminal capping ankyrin repeat of DarpMbp3_16
QDKFGKTAFDISIDNGNEDLAEILQKLN

SEQ ID NO: 11 - protein, 136 aa, Artificial
  DarpMbp3_16 M43C
MRGSHHHHHHGSDLGKKLLEAAHAGQDDEVRILMANGADVNACDNFGVTPLHLAAYWGHFEIVEVLLKYGADVNASDATGDTPLHLAAKWGYLGIVEVLLKYGADVNAQDKFGKTAFDISIDNGNEDLAEILQKLN

SEQ ID NO: 12 - protein, 136 aa, Artificial
  DarpMbp3_16 M45C
MRGSHHHHHHGSDLGKKLLEAAHAGQDDEVRILMANGADVNAMDCFGVTPLHLAAYWGHFEIVEVLLKYGADVNASDATGDTPLHLAAKWGYLGIVEVLLKYGADVNAQDKFGKTAFDISIDNGNEDLAEILQKLN

SEQ ID NO: 13 - protein, 136 aa, Artificial
  DarpMbp3_16 F46C
MRGSHHHHHHGSDLGKKLLEAAHAGQDDEVRILMANGADVNAMDNCGVTPLHLAAYWGHFEIVEVLLKYGADVNASDATGDTPLHLAAKWGYLGIVEVLLKYGADVNAQDKFGKTAFDISIDNGNEDLAEILQKLN

SEQ ID NO: 14 - protein, 136 aa, Artificial
  DarpMbp3_16 V48C
MRGSHHHHHHGSDLGKKLLEAAHAGQDDEVRILMANGADVNAMDNFGCTPLHLAAYWGHFEIVEVLLKYGADVNASDATGDTPLHLAAKWGYLGIVEVLLKYGADVNAQDKFGKTAFDISIDNGNEDLAEILQKLN

SEQ ID NO: 15 - protein, 136 aa, Artificial
  DarpMbp3_16 Y56C
MRGSHHHHHHGSDLGKKLLEAAHAGQDDEVRILMANGADVNAMDNFGVTPLHLAACWGHFEIVEVLLKYGADVNASDATGDTPLHLAAKWGYLGIVEVLLKYGADVNAQDKFGKTAFDISIDNGNEDLAEILQKLN

SEQ ID NO: 16 - protein, 136 aa, Artificial
  DarpMbp3_16 W57C
MRGSHHHHHHGSDLGKKLLEAAHAGQDDEVRILMANGADVNAMDNFGVTPLHLAAYCGHFEIVEVLLKYGADVNASDATGDTPLHLAAKWGYLGIVEVLLKYGADVNAQDKFGKTAFDISIDNGNEDLAEILQKLN

SEQ ID NO: 17 - protein, 136 aa, Artificial
  DarpMbp3_16 S76C
MRGSHHHHHHGSDLGKKLLEAAHAGQDDEVRILMANGADVNAMDNFGVTPLHLAAYWGHFEIVEVLLKYGADVNACDATGDTPLHLAAKWGYLGIVEVLLKYGADVNAQDKFGKTAFDISIDNGNEDLAEILQKLN

SEQ ID NO: 18 - protein, 136 aa, Artificial
  DarpMbp3_16 A78C
MRGSHHHHHHGSDLGKKLLEAAHAGQDDEVRILMANGADVNAMDNFGVTPLHLAAYWGHFEIVEVLLKYGADVNASDCTGDTPLHLAAKWGYLGIVEVLLKYGADVNAQDKFGKTAFDISIDNGNEDLAEILQKLN

SEQ ID NO: 19 - protein, 136 aa, Artificial
  DarpMbp3_16 T79C
MRGSHHHHHHGSDLGKKLLEAAHAGQDDEVRILMANGADVNAMDNFGVTPLHLAAYWGHFEIVEVLLKYGADVNASDACGDTPLHLAAKWGYLGIVEVLLKYGADVNAQDKFGKTAFDISIDNGNEDLAEILQKLN

SEQ ID NO: 20 - protein, 136 aa, Artificial
  DarpMbp3_16 D81C
MRGSHHHHHHGSDLGKKLLEAAHAGQDDEVRILMANGADVNAMDNFGVTPLHLAAYWGHFEIVEVLLKYGADVNASDATGCTPLHLAAKWGYLGIVEVLLKYGADVNAQDKFGKTAFDISIDNGNEDLAEILQKLN

SEQ ID NO: 21 - protein, 136 aa, Artificial
  DarpMbp3_16 K89C
MRGSHHHHHHGSDLGKKLLEAAHAGQDDEVRILMANGADVNAMDNFGVTPLHLAAYWGHFEIVEVLLKYGADVNASDATGDTPLHLAACWGYLGIVEVLLKYGADVNAQDKFGKTAFDISIDNGNEDLAEILQKLN

SEQ ID NO: 22 - protein, 136 aa, Artificial
  DarpMbp3_16 W90C
MRGSHHHHHHGSDLGKKLLEAAHAGQDDEVRILMANGADVNAMDNFGVTPLHLAAYWGHFEIVEVLLKYGADVNASDATGDTPLHLAAKCGYLGIVEVLLKYGADVNAQDKFGKTAFDISIDNGNEDLAEILQKLN

SEQ ID NO: 23 - protein, 43 aa, Artificial
  Consensus N-terminal capping AR
MRGSHHHHHHGSDLGKKLLEAARAGQDDEVRILMANGADVNAX

SEQ ID NO: 24 - protein, 25 aa, Artificial
  Consensus C-terminal capping AR
QDKFGKTAFDISIDNGNEDLAEILQ

SEQ ID NO: 25 - DNA, 3788 bp, Artificial
  pQEmbp3_161 vector sequence
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgatctgggtaaaaaactgctggaagctgcacacgctggccaagacgatgaagttcgtatcctgatggcgaacggtgcggatgtaaacgcaatggataactttggcgtgaccccactgcatctggctgcctactggggtcacttcgagattgtggaagtactgctgaaatatggtgcagacgtcaatgcctccgacgcaacgggtgacaccccgctgcacctggccgctaaatggggctacctgggtatcgttgaagttctgctgaagtacggcgcggacgttaacgcgcaggataaattcggtaaaactgctttcgacatctctattgataacggcaacgaagacctggcggagatcctgcagaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 26 - DNA, 3887 bp, Artificial
  pQEOFF7 plasmid
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgacctgggtaggaaactgctggaagctgctcgtgctggtcaggacgacgaagttcgtatcctgatggctaacggtgctgacgttaatgctgctgacaatactggtactactccgctgcacctggctgcttattctggtcacctggaaatcgttgaagttctgctgaagcacggtgctgacgttgacgcttctgacgtttttggttatactccgctgcacctggctgcttattggggtcacctggaaatcgttgaagttctgctgaagaacggtgctgacgttaacgctatggactctgatggtatgactccactgcacctggctgctaagtggggttacctggaaatcgttgaagttctgctgaagcacggtgctgacgttaacgctcaggacaaattcggtaagaccgctttcgacatctccatcgacaacggtaacgaggacctggctgaaatcctgcaaaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 27 - DNA, 510 bp, Artificial
  DarpOff7 coding sequence
atgagaggatcgcatcaccatcaccatcacggatccgacctgggtaggaaactgctggaagctgctcgtgctggtcaggacgacgaagttcgtatcctgatggctaacggtgctgacgttaatgctgctgacaatactggtactactccgctgcacctggctgcttattctggtcacctggaaatcgttgaagttctgctgaagcacggtgctgacgttgacgcttctgacgtttttggttatactccgctgcacctggctgcttattggggtcacctggaaatcgttgaagttctgctgaagaacggtgctgacgttaacgctatggactctgatggtatgactccactgcacctggctgctaagtggggttacctggaaatcgttgaagttctgctgaagcacggtgctgacgttaacgctcaggacaaattcggtaagaccgctttcgacatctccatcgacaacggtaacgaggacctggctgaaatcctgcaaaagcttaattag

SEQ ID NO: 28 - protein, 169 aa, Artificial
  DarpOff7 peptide sequence
MRGSHHHHHHGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 29 - DNA, 510 bp, Artificial
  Optimised DarpOff7 coding sequence
atgagaggatcgcatcaccatcaccatcacggatccgatctcgggcgcaaactgctggaggcagcccgagcaggccaagacgacgaagtgcgtattctgatggccaacggtgctgatgtcaacgctgcagataataccggcacgacaccactccatttggccgcgtatagtggccatttggaaattgtcgaagtactcttaaaacacggggcagatgtggatgcatcagacgtgttcggctacaccccgctgcatctggcggcatactggggtcacctggagattgttgaggttttgctgaagaatggcgctgatgttaacgcgatggatagcgatgggatgacgcctttgcacctcgcagccaagtgggggtacctcgaaatcgtagaagtcctgcttaagcatggtgcggacgtgaatgctcaggacaaatttggcaaaacagcgttcgatatcagcatcgataacggcaacgaggatctggcagaaattttacagaagcttaattag

SEQ ID NO: 30 - protein, 33 aa, Artificial
  Ankyrin repeat
NGRTPLHLAARNGHLEVVKLLLEAGADVNAKDK

SEQ ID NO: 31 - protein, 169 aa, Artificial
  DarpOff7 variant
MRGSHHHHHHGSDLGRKLLEAACAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 32 - protein, 169 aa, Artificial
  DarpOff7 variant
MRGSHHHHHHGSDLGRKLLEAARAGQDDEVRILMANGADVNAADCTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 33 - protein, 169 aa, Artificial
  DarpOff7 variant
MRGSHHHHHHGSDLGRKLLEAARAGQDDEVRILMANGADVNAADNCGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 34 - protein, 169 aa, Artificial
  DarpOff7 variant
MRGSHHHHHHGSDLGRKLLEAARAGQDDEVRILMANGADVNAADNTGTTPLHCAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 35 - protein, 169 aa, Artificial
  DarpOff7 variant
MRGSHHHHHHGSDLGRKLLEAARAGQDDEVRILMANGADVNAADNTGTTPLHLAAYSGHLEIVEVLLCHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 36 - protein, 169 aa, Artificial
  DarpOff7 variant
MRGSHHHHHHGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDCDGMTPLHLAAKWGYLEIVEVLLKHGADVNAQDKFGKTAFDISIDNGNEDLAEILQKLN

SEQ ID NO: 37 - protein, 169 aa, Artificial
  DarpOff7 variant
MRGSHHHHHHGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSCGMTPLHLAAKWGYLEIVEVLLKHGADVNAQDKFGKTAFDISIDNGNEDLAEILQKLN

SEQ ID NO: 38 - protein, 169 aa, Artificial
  DarpOff7 variant
MRGSHHHHHHGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGCTPLHLAAKWGYLEIVEVLLKHGADVNAQDKFGKTAFDISIDNGNEDLAEILQKLN

SEQ ID NO: 39 - protein, 169 aa, Artificial
  DarpOff7 variant
MRGSHHHHHHGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAACWGYLEIVEVLLKHGADVNAQDKFGKTAFDISIDNGNEDLAEILQKLN